(19) United States
(12) Patent Application Publication
(10) Pub. No.: US 2022/0218873 A1
(43) Pub. Date: Jul. 14, 2022

(54) HYALURONIC ACID AND GELATIN-CONTAINING FORMULATIONS

(71) Applicant: Pandorum Technologies Private Limited, Bengaluru (IN)

(72) Inventors: Tuhin BHOWMICK, New York, NY (US); Arun CHANDRU, Bengaluru (IN); Shivaram SELVAM, Hyderabad (IN); Parinita AGRAWAL, Bengaluru (IN); Midhun BEN THOMAS, Bengaluru (IN); Prayag BELLUR, Bengaluru (IN); Deepthi MENON, Bengaluru (IN)

(21) Appl. No.: 17/585,509

(22) Filed: Jan. 26, 2022

Related U.S. Application Data
(63) Continuation of application No. PCT/IN2020/050654, filed on Jul. 27, 2020.

(30) Foreign Application Priority Data
Jul. 26, 2019 (IN) 201941030372

(51) Int. Cl. A61L 27/38; B33Y 10/00; B33Y 70/10; B33Y 80/00; A61L 27/22; A61L 27/20

(52) U.S. Cl. CPC A61L 27/3834; B33Y 10/00; B33Y 70/10; A61K 35/28; A61L 27/222; A61L 27/20; A61L 2430/16; B33Y 80/00

(57) ABSTRACT

The present disclosure discloses embodiments of a formulation for application to the cornea, the formulation comprising a hyaluronic acid, a gelatin, and exosomes. In certain variations, the exosomes may be naive mesenchymal stem cell-derived exosomes, primed mesenchymal stem cell derived-exosomes, or corneal stromal stem cell derived-exosomes. In certain variations, the primed mesenchymal stem cell-derived exosomes are exosomes derived from mesenchymal stem cells primed with a corneal stromal stem cell derived-conditioned medium.

### CROSS-REFERENCE TO RELATED APPLICATION

This application is a Continuation of International Application No. PCT/IN2020/050654, filed on Jul. 27, 2020, which claims priority to Indian Application No. 201941030372, filed on Jul. 26, 2019. All applications are hereby incorporated by reference in their entirety.

### FIELD OF INVENTION

The present disclosure broadly relates to the field of bioengineered formulations, in general, and discloses a bio-ink formulation and bio-printed lenticule, and its applications in the bio-medical field.

### BACKGROUND OF INVENTION

The organ eye, in an organism represents the visual system and performs various photo-sensory functions. Cornea is the outermost layer of the eye appearing as a transparent membrane-like tissue. The primary function of the cornea is to help focus vision and it plays an important role in sight. Although it appears to possess a simplified tissue structure, this tissue is comprised of multiple layers.

The layers of the cornea are sequentially: Epithelium, Bowman's membrane, Stroma, Descemet's Membrane, and Endothelium. Each of these tissue layers comprise different types of cells. The maintenance of this tissue relies on a regular supply of nutrients from tear fluid from the aqueous humour.

Cornea can be affected by trauma, infection, and several diseases such as corneal abrasion, corneal dystrophy, corneal ulcer, corneal neovascularization, Fuchs' dystrophy, keratitis, and keratoconus, among others. These conditions can lead to temporary or complete blindness and are among the leading causes of blindness in the world.

Some of the commonly used procedures for the treatment of corneal diseases include laser surgery, corneal transplant surgery, anterior lamellar keratoplasty, endothelial lamellar keratoplasty, and the use of artificial corneas. These treatments involve the replacement of a part or whole of the cornea. The healing of cornea after these treatments is often compromised, and thus, research is on-going to find better and effective alternatives. More than 90% of the cornea is the stroma, a highly organized, transparent connective tissue maintained by keratocytes, quiescent mesenchymal cells of neural crest origin.

Corneal blindness is the fourth leading cause of blindness with numerous causative factors such as infectious keratitis, inflammatory disorders, inherited corneal epithelial-stromal dystrophies, degenerative conditions, and trauma-induced injuries. Corneal transplantation, which is the most common treatment modality, poses challenges in the form of high cost, transplant rejection, and imbalance between demand and supply of clinical-grade cadaveric donor corneas. Also, there is a problem of batch-to-batch variation in the donor corneas. Therefore, pressing measures are required to address the needs in the field of corneal treatments, and the present disclosure addresses the problem related to corneal blindness and corneal defect. Ulag et al. 2020; Euro Pol J. 2020; 133; 109744. 10.1016/j.eurpolymj.2020.109744 discloses an artificial 3D printed cornea, however, the published study does not provide the artificial cornea, which meets the desirable parameters like transmittance. Hence, there is a need to provide a better solution to address this problem prevalent in the field.

### SUMMARY OF INVENTION

In an aspect of the present disclosure, there is provided a bio-ink formulation comprising: (a) a modified hyaluronic acid having a molecular weight in the range of 30-300 kDa, and with a degree of substitution in the range of 10-80%; (b) a modified collagen peptide having a molecular weight in the range of 10-80 kDa, and with a degree of substitution in the range of 10-80%; and (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation.

In another aspect of the present disclosure, there is provided a bio-ink formulation comprising: (a) a modified hyaluronic acid having a molecular weight in the range of 30-300 kDa, and with a degree of substitution in the range of 10-75%; (b) a modified collagen having a molecular weight in the range of 200-300 kDa, and with a degree of substitution in the range of 10-75%; and (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation.

In another aspect of the present disclosure, there is provided a bio-ink formulation comprising: (a) a first polymer selected from the group consisting of modified hyaluronic acid, modified polyethylene glycol, modified polyvinyl alcohol, modified poly(N-isopropylacrylamide), modified alginate, silk, and modified silk; (b) a second polymer selected from the group consisting of collagen peptide, modified collagen peptide, collagen, and modified collagen; (c) a thickener selected from the group consisting of gelatin, modified cellulose, gellan gum, xanthum gum, polyethylene glycol, poloxamer, polyvinyl alcohol, and alginate, wherein the bio-ink formulation is having a viscosity in the range of 1690-5300 cP.

In another aspect of the present disclosure, there is provided a process for preparing the bio-ink formulation as described herein, said process comprising: (a) contacting a modified hyaluronic acid having a molecular weight in the range of 30-300 kDa, and with a degree of substitution in the range of 10-80%, and a modified collagen peptide having a molecular weight in the range of 10-80 kDa, and with a degree of substitution in the range of 10-80%; and gelatin having a bloom value in the range of 50-325, to obtain a first mixture; and (b) contacting the first mixture with a photo-activator to obtain the bio-ink formulation.

In another aspect of the present disclosure, there is provided a process for preparing the bio-ink formulation as described herein, said process comprising: (a) contacting a modified hyaluronic acid having a molecular weight in the range of 30-300 kDa and with a degree of substitution in the range of 10-80%, and a modified collagen having a molecular weight in the range of 200-300 kDa and with a degree of substitution in the range of 10-80%, and gelatin having a bloom value in the range of 50-325, to obtain a first mixture; and (b) contacting the first mixture with a photo-activator to obtain the bio-ink formulation.

In another aspect of the present disclosure, there is provided a bio-printed corneal lenticule comprising the bio-ink formulation as described herein.

In another aspect of the present disclosure, there is provided a bio-printed corneal lenticule comprising: (a) a modified hyaluronic acid having a molecular weight in the range of 30-300 kDa, and with a degree of substitution in the range of 10-80%, and having a weight percentage in the range of 0.2-10% with respect to the bio-printed corneal lenticule; (b) a modified collagen peptide having a molecular weight in the range of 10-80 kDa, and with a degree of substitution in the range of 10-80%, and having a weight percentage in the range of 1-25% with respect to the bio-printed corneal lenticule; and (c) gelatin having a bloom value in the range of 50-325, and having a weight percentage in the range of 0.01-15% with respect to the bio-printed corneal lenticule.

In another aspect of the present disclosure, there is provided a bio-printed corneal lenticule comprising: (a) a modified hyaluronic acid having a molecular weight in the range of 30-300 kDa, and with a degree of substitution in the range of 10-80%, and having a weight percentage in the range of 0.2-10% with respect to the bio-printed corneal lenticule; (b) a modified collagen having a molecular weight in the range of 200-300 kDa, and with a degree of substitution in the range of 10-75%, and having a weight percentage in the range of 5-50% with respect to the bio-printed corneal lenticule; and (c) gelatin having a bloom value in the range of 50-325, and having a weight percentage in the range of 0.01-15% with respect to the bio-printed corneal lenticule.

In another aspect of the present disclosure, there is provided a bio-printed corneal lenticule comprising: (a) a first polymer selected from the group consisting of modified hyaluronic acid, modified polyethylene glycol, modified polyvinyl alcohol, modified poly(N-isopropylacrylamide), modified alginate, silk, and modified silk; (b) a second polymer selected from the group consisting of collagen peptide, modified collagen peptide, collagen, and modified collagen; (c) a thickener selected from the group consisting of gelatin, modified cellulose, gellan gum, xanthum gum, polyethylene glycol, poloxamer, polyvinyl alcohol, and alginate.

In another aspect of the present disclosure, there is provided a bio-printed corneal lenticule comprising: (a) a first polymer selected from the group consisting of modified hyaluronic acid, modified polyethylene glycol, modified polyvinyl alcohol, modified poly(N-isopropylacrylamide), modified alginate, silk, and modified silk; (b) a second polymer selected from the group consisting of collagen peptide, modified collagen peptide, collagen, and modified collagen; (c) a thickener selected from the group consisting of gelatin, modified cellulose, gellan gum, xanthum gum, polyethylene glycol, poloxamer, polyvinyl alcohol, and alginate; and (d) exosomes selected from the group consisting of naive mesenchymal stem cell-derived exosomes, primed mesenchymal stem cell derived-exosomes, and corneal stromal stem cell derived-exosomes.

In another aspect of the present disclosure, there is provided a bio-printed corneal lenticule comprising: (a) a first polymer selected from the group consisting of modified hyaluronic acid, modified polyethylene glycol, modified polyvinyl alcohol, modified poly(N-isopropylacrylamide), modified alginate, silk, and modified silk; (b) a second polymer selected from the group consisting of collagen peptide, modified collagen peptide, collagen, and modified collagen; (c) a thickener selected from the group consisting of gelatin, modified cellulose, gellan gum, xanthum gum, polyethylene glycol, poloxamer, polyvinyl alcohol, and alginate; and (d) stem cells selected from the group consisting of human corneal stromal stem cells, human corneal limbal stem cells, human bone marrow-derived mesenchymal stem cells, adipose tissue-derived mesenchymal stem cells, umbilical cord-derived mesenchymal stem cells, Wharton jelly-derived mesenchymal stem cells, dental pulp derived mesenchymal stem cells, placental mesenchymal stem cells, and induced pluripotent stem cells.

In another aspect of the present disclosure, there is provided a process for obtaining a bio-printed corneal lenticule, said process comprising: (a) obtaining a bio-ink formulation as described herein; (b) printing the bio-ink formulation over a scaffold to obtain a printed corneal structure; and (c) exposing the printed corneal structure to a light having a wavelength in the range of 420-570 nm, and having an intensity in the range of 50-150 mW/cm² for a time period in the range of 1-15 minutes for obtaining the bio-printed corneal lenticule.

In another aspect of the present disclosure, there is provided a bio-printed corneal lenticule obtained by the process as described herein.

In another aspect of the present disclosure, there is provided a method for treating a corneal defect in a subject, said method comprises: (a) obtaining the bio-printed corneal lenticule as described herein; and (b) implanting the bio-printed corneal lenticule at the site of the corneal defect, for treating the corneal defect in the subject.

In another aspect of the present disclosure, there is provided a bio-printed corneal lenticule as described herein for use in treating corneal defects in a subject.

In another aspect of the present disclosure, there is provided a bio-printed corneal lenticule as described herein for use in in-vitro drug toxicity studies and disease modelling.

In another aspect of the present disclosure, there is provided a bio-ink formulation as described herein, for use in obtaining a bio-printed corneal lenticule.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIGS. 1A-1C depict the schematic of bio-ink formulation prepared by the method as disclosed in the present disclosure for preparing bio-printed corneal lenticule. FIG. 1A depicts the general protocol. FIG. 1B depicts a modified protocol where photo-crosslinker (eosin) solution is added in two steps. FIG. 1C depicts a thickener based bio-ink preparation in accordance with an embodiment of the present disclosure.

FIG. 2 depicts the viscosity assessment of solutions with different molecular weight, concentration and degree of substitution (DoS) of HA-MA, in accordance with an embodiment of the present disclosure.

FIG. 3 depicts the viscosity assessment of bio-ink with A) different concentration of 250 kDa HA-MA (30% DoS) with 50 mg/ml RCP-SH (DoS 50%), and two modes of addition of photo-initiator (eosin), in accordance with an embodiment of the present disclosure.

FIG. 4 depicts the viscosity assessment of bio-ink using methyl cellulose and gelatin as thickeners in combination with the HA-MA (250 kDa, 30% DoS and 50 kDa, 50% DoS) and RCP-SH (80 mg/ml, 50% DoS) as base polymers, in accordance with an embodiment of the present disclosure.

FIG. 5 depicts the schematic of bioprinting process using the bio-ink as described in the present disclosure, and cells to develop a bioengineered corneal stroma, in accordance with an embodiment of the present disclosure.

FIGS. 6A-6C depict printability assessments of the bioink with methyl cellulose and gelatin as thickeners. The dimensions of the printed lenticule as shown in the figures are 400 microns thick and 14 mm in diameter, in accordance with an embodiment of the present disclosure. FIG. 6D depicts a representation of flow requirements for bioprinting.

FIG. 7 depicts the compression modulus of solutions with different concentration of 250 kDa HA-MA (30% DoS) with 50 mg/ml RCP-SH, and two modes of the addition of photo-initiator (eosin). In addition, the effect of the addition of thickener (60 mg/ml gelatin) on compression modulus of the 50 kDa HA-MA/RCP-SH (35/150 mg/mL, both DoS 50%) hydrogel has been demonstrated, in accordance with an embodiment of the present disclosure.

FIG. 8 depicts the visible light transmittance by the hydrogels HA-MA/RCP-SH (35/150 mg/mL, both DoS 50%) in PBS. Data is represented as mean±SD for three replicate samples, in accordance with an embodiment of the present disclosure.

FIG. 9 depicts the swelling profile of bioprinted lenticule HA-MA/RCP-SH (35/150 mg/mL, both DoS 50%) with respect to time. Data is represented as mean±SD for three replicate samples, in accordance with an embodiment of the present disclosure.

FIG. 10 depicts the gelatin release profile from the bioprinted lenticules HA-MA/RCP-SH (35/150 mg/mL, both DoS 50%) with respect to time. (n=3, ±SD), in accordance with an embodiment of the present disclosure.

FIG. 11 depicts the biodegradation of the lenticule in PBS. Data represents mean±SD with n=3 replicates, in accordance with an embodiment of the present disclosure.

FIG. 12 depicts the cell viability study for bioprinted hydrogel formulation (50 kDa HA-MA 35 mg/ml+RCP-SH 150 mg/ml, both DoS 50%) with CLSCs encapsulated bioink. Cells on coverslips were cultured on the surface. Scale bar=200 in accordance with an embodiment of the present disclosure.

FIG. 13 depicts the cell viability study for bioprinted hydrogel formulation (50 kDa HA-MA 35 mg/ml+RCP-SH 150 mg/ml, both DoS 50%) with BM-MSCs encapsulated bioink. Bottom panel depicts the homogenous distribution of cells inside in the bioprinted hydrogel. Scale bar=200 in accordance with an embodiment of the present disclosure.

FIGS. 14A-14B depict the immunofluorescence study showing expression of CD90 (red, FIG. 14A) and αSMA (green, FIG. 14B) by the CLSCs encapsulated in the bioprinted lenticule (50 kDa HA-MA 35 mg/ml+RCP-SH 150 mg/ml, both DoS 50%) with respect to the 2D culture surface. Scale bar=100 in accordance with an embodiment of the present disclosure.

FIG. 15 depicts the light transmittance study of 50 kDa HA-MA (50% DoS)/Col-MA bioink in 50/9 mg/ml concentration ratio and its individual components in visible light, in accordance with an embodiment of the present disclosure.

FIG. 16 depicts the cell viability study for CLSCs encapsulated in 50 kDa HA-MA (50% DoS)/Col-MA bioink in 50/9 mg/ml concentration ratio. Scale bar=50 μm, in accordance with an embodiment of the present disclosure.

FIGS. 17A-17B depict the biomarker expression CD90 (red) and αSMA (green) by CLSCs encapsulated in a representative Pandorum's bioink (50 kDa HA-MA/250 kDa ColMA, 50/9 mg/ml, FIG. 17A) or a 2D surface (FIG. 17B), reflecting cell phenotype with progress in culture duration. Scale bar=50 μm, in accordance with an embodiment of the present disclosure.

FIGS. 18A-18H depict the phase contrast microscopy image depicting epithelialization of the 2D coverslip, Gel-MA (200 mg/ml, DoS >95%), “33 kDa” HA-MA/RCP-SH (75/125 and 75/150 mg/ml, both DoS 50%) hydrogel surface with primary human corneal epithelial cells on day 3 and 13 in vitro (scale bar=100 μm), in accordance with an embodiment of the present disclosure.

FIGS. 19A-19H depict the cell viability study for CLSCs cultured on the “33 kDa” HA-MA/RCP-SH (mg/ml, both DoS 50%) hydrogel surface and Gel-MA (200 mg/ml, DoS >95%) and 2D culture surfaces. (Scale bar=500 μm), in accordance with an embodiment of the present disclosure.

FIG. 20 depicts the cell viability study for the CLSCs encapsulated in the “33 kDa” HA-MA/RCP-SH (mg/ml, both DoS 50%) hydrogels and Gel-MA (200 mg/ml, DoS >95%). Cells on coverslips were cultured on the surface, in accordance with an embodiment of the present disclosure.

FIG. 21A depicts the immunofluorescence study showing expression of CD90 (red)by the CLSCs encapsulated in a representative “33 kDa” HA-MA/RCP-SH (both

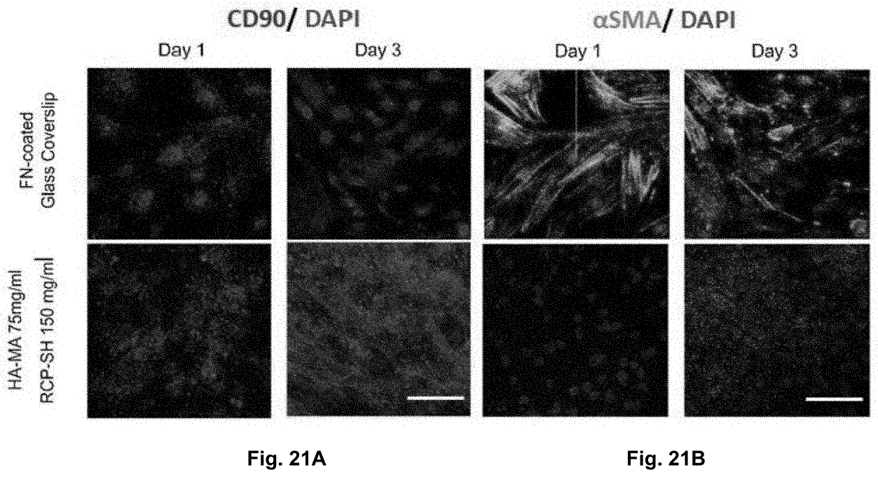

DoS 50%) hydrogel formulation with respect to the 2D culture surface. (Scale bar=100 μm), in accordance with an embodiment of the present disclosure. FIG. 21B depicts the immunofluorescence study showing expression of and αSMA (green) by the CLSCs encapsulated in a representative “33 kDa” HA-MA/RCP-SH (both DoS 50%) hydrogel formulation with respect to the 2D culture surface. (Scale bar=100 μm), in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### DEFINITIONS

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, concentration in a range of 2-100 mg/ml range of about 2-100 should be interpreted to include not only the explicitly recited limits of about 2 to about 100, but also to include sub-ranges, such as 10-90, 25-75, and so forth, as well as individual amounts, including fractional amounts, within the specified ranges, such as 35.5, and 45.5, for example.

The term “decellularized extra cellular matrix (dECM)” refers to a biomaterial which is obtained after decellularization of a specific type of cell population. The dECM can be cell culture-derived dECM in which the specific type of cell population is obtained by in-vitro cell culturing methods. Some examples of the cell secreted ECM components of interest are lumican, decorin, keratocan. The term “cell-derived component” refers to any component or a combination of components which are derived from cells. The cell-derived component is generally obtained from conditioned medium which comprises exosomes, cell modulators, secreted factors and other components. The term “conditioned medium” refers to the media enriched with cell secreted factors such as various proteins/growth factors, such as hepatocyte growth factor (HGF), keratocyte growth factor (KGF) and soluble form like tyrosine kinase 1 (sFLT1), Pigment epithelial-derived growth factor (PEDF) ,thrombospondin and exosomes containing various molecules including miR-10b, miR-21, miR-23a, miR-182, miR-181a, miR-145 and epidermal growth factor (EGF), fibroblast growth factor (FGF), sFLT1 and phosphoglycerate kinase (PGK), phosphoglucomutase, enolase, CD73, CD63 and MMP9. The composition of conditioned medium is intended to be exploited for therapeutic applications. The term “cell modulators” refers to various secreted factors such as ECM, growth factors, exosomal cargos containing a broad range of small and macromolecules, many of protein or nucleic acid in nature. Some of these include micro-RNA, mRNA, long non-coding RNA, lipid mediator, that can modulate cellular response. The term “exosomes” refers to cell-secreted vesicles containing cargo molecules of protein or nucleic acid in nature, often referring to the 20-200 nm range with molecules of clinical interest such as, anti-inflammatory, anti-fibrotic and regenerative properties.

The term “bio-ink formulation” is used to mean a formulation/composition comprising the components as disclosed herein. The bio-ink formulation denotes a formulation that is used as an ink for printing a bio-printed corneal lenticule using a 3D printer.

The term “bio-printed corneal lenticule” or “bio-printed lenticule” is referred to a synthetic material which is obtained by printing the bio-ink formulation as disclosed herein on a scaffold using a 3D printer. The dimensions of the lenticule may vary as per the requirements of the subject in need thereof. The lenticule can be used for replacing the entire damaged cornea or can be made as per the area required to be repaired on the cornea.

The bio-ink formulation as disclosed in the present disclosure is a mixture of polymers which are not cross-linked completely in the formulation. As per few implementations, the bio-ink comprise a photo-initiator which start the cross-linking process in the presence of light, however, for complete cross-linking to take place, an exposure to high intensity while light is required as disclosed in the present disclosure. The complete cross-linked bio-ink can also be referred to as “hydrogel”. As a person skilled in the art would understand that testing of certain parameters like compressive modulus and tensile strength would only be possible in the cross-linked product like hydrogel. Also, the bio-printed corneal lenticule is a product which is obtained by printing the bio-ink formulation on a scaffold, followed by exposure to high-intensity white light for complete cross-linking to take place. Also, testing of certain parameters can only be done on the bio-printed product to assess the usefulness of the corresponding bio-ink formulation.

The term “corneal defect” or “corneal disorder” have been used interchangeably to denote the issues in the cornea which require medical intervention. The intervention can be to the extent of replacing the damaged corneal with the bio-printed lenticule as described in the present disclosure.

The terms “collagen” and “collagen sequence derived peptide” as used herein is used to include natural, synthetic, recombinant and/or alternate versions of said polypeptide and protein sequences.

The term “modified hyaluronic acid” or “modified collagen peptide” or “modified collagen”, or “modified silk” or “modified cellulose” or “polyethylene glycol” or “modified polyvinyl alcohol” or “modified alginate” denotes any kind of modification that is possible in the respective molecules. The specific modifications that have been done are covered in the presented disclosure. For example, modified cellulose intend to mean the modified molecules like methyl cellulose, carboxymethyl cellulose (CMC), hydroxypropyl methyl cellulose (HPMC), and hydroxyethyl methyl cellulose (HEMC).

The term “mesenchymal stem cell derived-conditioned medium or “MSC-CM” refers to the medium obtained after the growth of the MSC. The conditioned medium thus obtained comprises secreted cell modulators and multiple factors critical for tissue regeneration. The conditioned medium thus obtained also comprises secretome, and exosomes which need to be purified from the conditioned medium before being able to apply for therapeutic purposes. The process for obtaining expanded MSC as described herein also leads to the formation of MSC-CM, therefore, it can be said that a single process leads to the procurement of a population of expanded MSC as well as of MSC-CM. The term “exosomes” refers to the type of an extracellular vesicle that contains constituents (in terms of protein, DNA, and RNA) of the biological cells that secretes them. The exosomes obtained from the conditioned medium as described herein is used for therapeutic purposes.

The term “corneal stromal stem cell derived-conditioned medium or “CSSC-CM” refers to the medium in which corneal stromal stem cells (CSSC) are grown. The CSSC-CM as described herein is obtained by culturing of CSSC in a manner known in the art or by culturing of CSSC as per the method disclosed herein. Corneal limbal Stem Cells (CLSC) are isolated from the limbal ring as described in previous PCT Applications; PCT/IN2020/050622 & PCT/IN2020/050623. These cells can be divided into two subpopulations: corneal stromal stem cells (CSSC) and Limbal Epithelial Stem Cells (LESC). The PCT Application PCT/IN2020/050622 & PCT/IN2020050623 disclose methods for CSSC isolation and demonstrates enrichment of CSSC population over LESCs by the protocol used therein. However, in case there is a small population of LESCs left behind in the CSSC enriched fraction, the same is being referred to as ‘CLSC’ to cover all cell types in these applications. Therefore, the conditioned medium derived from such CSSC enriched population is known as CSSC-derived conditioned medium (CSSC-CM). It is understood that for the sake of simplicity, the term CSSC-CM is also used to denote the conditioned medium obtained by culturing enriched CSSC in which a small population of LESC is also present.

The term “xeno-free” as described in the present disclosure, refers to the process as described herein, which is free of any product which is derived from a non-human animal. The method being xeno-free is an important advantage because of its plausibility of clinical application. The term “scalable” refers to the ability to increase the production output manifolds. The term “subject” refers to a human subject or a mammalian subject who is suffering from the conditions as mentioned in the present disclosure. The term “therapeutically effective amount” refers to the amount of a composition which is required for treating the conditions of a subject.

The term “scaffold” refers to a mold or an inert substance that is used as a support on which the bio-ink is printed. As per an implementation of the present disclosure, the printing is done using a 3D printer.

The term “culture medium” refers to the medium in which the MSC is cultured. The culture medium comprises MSC basal medium, and the MSC basal medium is used as per the MSC, which is being cultured. The MSC basal medium as mentioned in the present disclosure, was commercially procured. For the purposes of the present disclosure, RoosterBio xenofree media was used for BMMSCs.

Partial or complete corneal implants are one of the most successful therapies for the treatment of corneal diseases. The present disclosure provides a solution to the problem associated with sub-par healing of the cornea after treatment of corneal diseases by various means by providing an effective and efficient bioengineered bio-printed corneal lenticule. The present disclosure discloses a bio-ink formulation comprising: (a) polymers selected from the group consisting of collagen (methacrylated and thiolated), collagen peptide derivatives, hyaluronic acid and its modifications (methacrylated and thiolated), cellulose derivatives (methyl cellulose, carboxymethyl cellulose, and their methacrylated and thiolated derivatives), polyethylene glycol derivatives (linear and multi-arm; methacrylated and thiolated), polyvinyl alcohol (methacrylated and thiolated), gelatin (methacrylated and thiolated), chitosan, and alginate; and (b) thickener selected from the group consisting of gelatin, gellan gum, xanthum gum, cellulose derivatives, such as methyl cellulose, carboxymethyl cellulose (CMC), hydroxypropyl methyl cellulose (HPMC) and hydroxyethyl methyl cellulose (HEMC), polyethylene glycol, poloxamer, polyvinyl alcohol, and alginate. The bio-ink formulation is formulated to have an optimum viscosity so that it can be easily printed using a 3D printer machine, to obtain a bio-printed corneal lenticule. The bio-printed corneal lenticule can further be used for treating corneal defects in the subjects. The bio-ink formulation, as well as the bio-printed corneal lenticule, are xeno-free, and scalable to satisfy the clinical requirements of corneal implants. The bio-printed corneal lenticule was obtained with an optical thickness in the range of 5-500 microns.

The PCT Application PCT/IN2020/050622 and PCT/IN2020050623 were filed from the Applicant of the present disclosure and disclose two-dimensional, and three-dimensional methods of culturing stem cells and expanded stem cells and stem cell-derived conditioned medium. The above-mentioned PCT Applications also disclose methods for obtaining expanded primed mesenchymal stem cells and conditioned medium derived from the expanded primed mesenchymal stem cells. The PCT Application No. PCT/IN2020/050622 and PCT/IN2020050623 are incorporated herein in entirety.

The challenges raised by the current treatment modality can be addressed through the use of biomaterials and incorporation of adult stem cells within it by employing the 3D bioprinting technique. In support of the same, the present disclosure also discloses a bio-ink formulation comprising stem cells selected from the group consisting of human corneal stromal stem cells, human corneal limbal stem cells, human bone marrow-derived mesenchymal stem cells, adipose tissue-derived mesenchymal stem cells, umbilical cord-derived mesenchymal stem cells, Wharton jelly-derived mesenchymal stem cells, dental pulp derived mesenchymal stem cells, and induced pluripotent stem cells. Further, the present disclosure also discloses the bio-ink formulation comprising exosomes selected from the group consisting of naive mesenchymal stem cell-derived exosomes, primed mesenchymal stem cell derived-exosomes, and corneal stromal stem cell derived-exosomes. The presence of exosomes in the bio-printed lenticule can help in the treatment of corneal disorders by virtue of the regenerative potential of the exosomes. The present disclosure also discloses the bio-ink formulation comprising stem cells as well as exosomes, which can further enhance the therapeutic potential of the bio-printed corneal lenticule.

The bio-ink formulation with or without stem cells or exosomes, along with the photo-initiator crosslinks in the presence of light to yield a bio-printed corneal lenticule which is transparent. The bio-printed corneal lenticule is biomimetic as it possesses the physical, mechanical and biological properties that match the characteristics of native cornea tissue. Also, the bio-ink formulation is biocompatible and possesses cornea-mimetic properties and promotes human corneal epithelial cell migration as well as proliferation.

The development of a transparent suturable 3D bio-printed corneal lenticule using hyaluronic acid, which is a natural component present in the eye can serve as a viable therapeutic option for replacement of diseased/injured corneas with partial or full-thickness transplant grafts.

The following paragraphs depict the embodiments of the claimed bioengineered corneal stromal composition and synthetic corneal stroma. Additionally, the processes for preparing said bioengineered corneal stromal composition and the synthetic corneal stroma are also depicted. Also provided are the bio-ink compositions that are used to manufacture the claimed bioengineered corneal stromal composition and synthetic corneal stroma. However, a person skilled in the art can employ conditions as per his need and prepare the composition based on the representative examples, and such processes would fall within the scope of the present invention.

The embodiments further depict the bioengineered corneal stromal composition as disclosed herein, comprising at least one extra cellular matrix (ECM)-mimetic polymer, and at least one crosslinker polymer.

The present disclosure is not to be limited in scope by the specific embodiments described herein, which are intended for the purposes of exemplification only. Functionally-equivalent products, compositions, and methods are clearly within the scope of the disclosure, as described herein.

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

In an embodiment of the present disclosure, there is provided a bio-ink formulation comprising: (a) a modified hyaluronic acid having a molecular weight in the range of 30-300 kDa, and with a degree of substitution in the range of 10-80%; (b) a modified collagen peptide having a molecular weight in the range of 10-80 kDa, and with a degree of substitution in the range of 10-80%; and (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation. In another embodiment of the present disclosure, the modified hyaluronic acid has a molecular weight in the range of 30-300 kDa, or 40-280 kDa, or 40-250 kDa, or 40-200 kDa, or 40-150 kDa, or 40-125 kDa, or 40-100 kDa, or 40-75 kDa, or 40-60 kDa, and wherein the degree of substitution of the modified hyaluronic acid is in the range of 20-70%, or 30-65%, or 35-60%, or 40-60%, and wherein the modified collagen peptide has a molecular weight in the range of 20-70 kDa, or 25-65 kDa, or 30-60 kDa, or 35-55 kDa, or 40-55 kDa, or 45-55 kDa, and wherein the degree of substitution of the modified collagen peptide is in the range of 20-70%, or 30-65%, or 35-60%, or 40-60%, and wherein the gelatin has a bloom value in the range of 75-300, or 100-275, or 125-250, or 175-225, and wherein gelatin has a concentration in the range of 50-80 mg/ml, or 55-75 mg/ml, or 55-70 mg/ml, or 55-65 mg/ml, or 0.5-120 mg/ml, or 5-120 mg/ml, or 15-100 mg/ml, or 25-90 mg/ml, or 40-90 mg/ml.

In an embodiment of the present disclosure, there is provided a bio-ink formulation comprising: (a) a modified hyaluronic acid having a molecular weight of 50 kDa, and with a degree of substitution of 50%; (b) a modified collagen peptide having a molecular weight of 50 kDa, and with a degree of substitution of 50%; and (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation.

In an embodiment of the present disclosure, there is provided a bio-ink formulation comprising: (a) a modified hyaluronic acid having a molecular weight of 50 kDa, and with a degree of substitution in the range of 30-70%; (b) a modified collagen peptide having a molecular weight of 50 kDa, and with a degree of substitution in the range of 30-70%; and (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation.

In an embodiment of the present disclosure, there is provided a bio-ink formulation comprising: (a) a modified hyaluronic acid having a molecular weight of 50 kDa, and with a degree of substitution of 50% having a concentration range of 2-100 mg/ml with respect to the bio-ink formulation; (b) a modified collagen peptide having a molecular weight of 50 kDa, and with a degree of substitution of 50% having a concentration range of 10-250 mg/ml with respect to the bio-ink formulation; and (c) gelatin having a bloom value in the range of 50-325, wherein gelatin is in the concentration range of 50-100 mg/ml with respect to the bio-ink formulation.

In an embodiment of the present disclosure, there is provided a bio-ink formulation comprising: (a) a modified hyaluronic acid having a molecular weight of 50 kDa, and with a degree of substitution of 50% having a concentration range of 31-50 mg/ml with respect to the bio-ink formulation; (b) a modified collagen peptide having a molecular weight of 50 kDa, and with a degree of substitution in the range of 50% having a concentration range of 80-200 mg/ml with respect to the bio-ink formulation; and (c) gelatin having a bloom value in the range of 50-325, wherein gelatin is in the concentration range of 50-100 mg/ml with respect to the bio-ink formulation.

In an embodiment of the present disclosure, there is provided a bio-ink formulation comprising: (a) a modified hyaluronic acid having a molecular weight of 50 kDa, and with a degree of substitution in the range of 10-75%, preferably 50%; (b) a modified collagen having a molecular weight of 250 kDa, and with a degree of substitution in the range of 10-75%, preferably 29%; and (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation.

In an embodiment of the present disclosure, there is provided a bio-ink formulation comprising: (a) a modified hyaluronic acid having a molecular weight of 33 kDa, and with a degree of substitution in the range of 30-70%; (b) a modified collagen peptide having a molecular weight of 50 kDa, and with a degree of substitution in the range of 30-70%; and (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation.

In an embodiment of the present disclosure, there is provided a bio-ink formulation comprising: (a) a modified hyaluronic acid having a molecular weight of 33 kDa, and with a degree of substitution of 50%; (b) a modified collagen peptide having a molecular weight of 50 kDa, and with a degree of substitution of 50%; and (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation.

In an embodiment of the present disclosure, there is provided a bio-ink formulation comprising: (a) a modified hyaluronic acid having a molecular weight of 33 kDa, and with a degree of substitution of 50% having a concentration range of 2-100 mg/ml with respect to the bio-ink formulation; (b) a modified collagen peptide having a molecular weight of 50 kDa, and with a degree of substitution of 50% having a concentration range of 10-250 mg/ml with respect to the bio-ink formulation; and (c) gelatin having a bloom value in the range of 50-325, wherein gelatin is in the concentration range of 50-100 mg/ml with respect to the bio-ink formulation.

In an embodiment of the present disclosure, there is provided a bio-ink formulation comprising: (a) a modified hyaluronic acid having a molecular weight of 33 kDa, and with a degree of substitution of 50% having a concentration range of 31-50 mg/ml with respect to the bio-ink formulation; (b) a modified collagen peptide having a molecular weight of 50 kDa, and with a degree of substitution of 50% having a concentration range of 80-200 mg/ml with respect to the bio-ink formulation; and (c) gelatin having a bloom value in the range of 50-325, wherein gelatin is in the concentration range of 50-100 mg/ml with respect to the bio-ink formulation.

In an embodiment of the present disclosure, there is provided a bio-ink formulation comprising: (a) a modified hyaluronic acid having a molecular weight in the range of 30-300 kDa, and with a degree of substitution in the range of 10-75%; (b) a modified collagen having a molecular weight in the range of 200-300 kDa, and with a degree of substitution in the range of 10-75%; and (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation. In another embodiment of the present disclosure, the modified hyaluronic acid has a molecular weight in the range of 30-300 kDa, or 40-280 kDa, or 40-250 kDa, or 40-200 kDa, or 40-150 kDa, or 40-125 kDa, or 40-100 kDa, or 40-75 kDa, or 40-60 kDa, and wherein the degree of substitution of the modified hyaluronic acid is in the range of 20-70%, or 30-65%, or 35-60%, or 40-60%, and wherein the modified collagen has a molecular weight in the range of 210-280 kDa, or 225-260 kDa, or 235-250 kDa, and wherein the degree of substitution of the modified collagen is in the range of 20-70%, or 30-65%, or 35-60%, or 40-60%, and wherein the gelatin has a bloom value in the range of 75-300, or 100-275, or 125-250, or 175-225, and wherein gelatin has a concentration in the range of 50-80 mg/ml, or 55-75 mg/ml, or 55-70 mg/ml, or 55-65 mg/ml.

In an embodiment of the present disclosure, there is provided a bio-ink formulation comprising: (a) a first polymer selected from the group consisting of modified hyaluronic acid, modified polyethylene glycol, modified polyvinyl alcohol, modified poly(N-isopropylacrylamide), modified alginate, silk, and modified silk; (b) a second polymer selected from the group consisting of collagen peptide, modified collagen peptide, collagen, and modified collagen; (c) a thickener selected from the group consisting of gelatin, modified cellulose, gellan gum, xanthum gum, polyethylene glycol, poloxamer, polyvinyl alcohol, and alginate, wherein the bio-ink formulation is having a viscosity in the range of 1690-5300 cP. In another embodiment of the present disclosure, the viscosity of the bio-ink formulation is in the range of 1700-5000 cP, or 1800-4900 cP, or 1900-4800 cP, or 2000-4500 cP.

In an embodiment of the present disclosure, there is provided a bio-ink formulation comprising: (a) a modified hyaluronic acid having a molecular weight in the range of 30-300 kDa, and with a degree of substitution in the range of 10-80%; (b) a modified collagen peptide having a molecular weight in the range of 10-80 kDa, and with a degree of substitution in the range of 10-80%; and (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation, wherein the modified hyaluronic acid is in the concentration range of 2-100 mg/mL with respect to the bio-ink formulation, and wherein the modified collagen peptide is in a concentration range of 10-250 mg/ml with respect to the bio-ink formulation. In another embodiment of the present disclosure, the modified hyaluronic acid is in the concentration range of 5-90 mg/mL, or 10-80 mg/mL, or 15-80 mg/mL, or 20-70 mg/mL, or 25-70 mg/mL, or 30-60 mg/mL, or 30-55 mg/mL, 30-50 mg/mL, or 30-47 mg/mL with respect to the bio-ink formulation, and wherein the modified collagen peptide is in the concentration range of 20-230 mg/ml, or 50-200 mg/ml, or 75-200 mg/ml, or 90-200 mg/ml, or 100-200 mg/ml, or 125-175 mg/ml.

In an embodiment of the present disclosure, there is provided a bio-ink formulation comprising: (a) a modified hyaluronic acid having a molecular weight in the range of 30-300 kDa, and with a degree of substitution in the range of 10-75%; (b) a modified collagen having a molecular weight in the range of 200-300 kDa, and with a degree of substitution in the range of 10-75%; and (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation, and wherein the modified hyaluronic acid is in a concentration range of 2-100 mg/mL with respect to the bio-ink formulation, and the modified collagen is in a concentration range of 0.1-100 mg/ml with respect to the bio-ink formulation. In another embodiment of the present disclosure, the modified hyaluronic acid is in the concentration range of 5-90 mg/mL, or 10-80 mg/mL, or 15-80 mg/mL, or 20-70 mg/mL, or 25-70 mg/mL, or 30-60 mg/mL, or 30-55 mg/mL, 30-50 mg/mL, or 30-47 mg/mL with respect to the bio-ink formulation, and wherein the modified collagen is in a concentration range of 0.5-90 mg/ml, or 1-80 mg/ml, or 5-70 mg/ml, or 7-60 mg/ml, or 8-50 mg/ml, or 8-40 mg/ml, or 8-30 mg/ml, or 8-20 mg/ml.

In an embodiment of the present disclosure, there is provided a bio-ink formulation comprising: (a) a modified hyaluronic acid having a molecular weight in the range of 30-300 kDa, and with a degree of substitution in the range of 10-80%; (b) a modified collagen peptide having a molecular weight in the range of 10-80 kDa, and with a degree of substitution in the range of 10-80%; and (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation, and wherein the modified hyaluronic acid is selected from the group consisting of methacrylated hyaluronic acid, and thiolated hyaluronic acid, and wherein the modified collagen peptide is selected from the group consisting of thiolated collagen peptide, and methacrylated collagen peptide. In another embodiment of the present disclosure, the modified hyaluronic acid is methacrylated hyaluronic acid, and the modified collagen peptide is thiolated collagen peptide.

In an embodiment of the present disclosure, there is provided a bio-ink formulation comprising: (a) a modified hyaluronic acid having a molecular weight in the range of 30-300 kDa, and with a degree of substitution in the range of 10-75%; (b) a modified collagen having a molecular weight in the range of 200-300 kDa, and with a degree of substitution in the range of 10-75%; and (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation, and wherein the modified hyaluronic acid is selected from the group consisting of methacrylated hyaluronic acid, and thiolated hyaluronic acid, and wherein the modified collagen is selected from the group consisting of thiolated collagen, and methacrylated collagen. In another embodiment of the present disclosure, the modified hyaluronic acid is methacrylated hyaluronic acid, and the modified collagen is thiolated collagen.

In an embodiment of the present disclosure, there is provided a bio-ink formulation comprising: (a) a first polymer selected from the group consisting of modified hyaluronic acid, modified polyethylene glycol, modified polyvinyl alcohol, modified poly(N-isopropylacrylamide), modified alginate, silk, and modified silk; (b) a second polymer selected from the group consisting of collagen peptide, modified collagen peptide, collagen, and modified collagen; (c) a thickener selected from the group consisting of gelatin, modified cellulose, gellan gum, xanthum gum, polyethylene glycol, poloxamer, polyvinyl alcohol, and alginate, wherein the bio-ink formulation is having a viscosity in the range of 1690-5300 cP, and wherein the modified hyaluronic acid is selected from the group consisting of methacrylated hyaluronic acid, and thiolated hyaluronic acid, and wherein the modified collagen peptide is selected from the group consisting of thiolated collagen peptide, and methacrylated collagen peptide, and wherein the modified collagen is selected from the group consisting of thiolated collagen, and methacrylated collagen. In another embodiment of the present disclosure, the modified hyaluronic acid is methacrylated hyaluronic acid, and the modified collagen peptide is thiolated collagen peptide, and the modified collagen is thiolated collagen. In yet another embodiment of the present disclosure, the modified hyaluronic acid is in a concentration range of 2-100 mg/mL with respect to the bio-ink formulation, and the modified collagen peptide is in a concentration range of 10-250 mg/ml with respect to the bio-ink formulation, and the modified collagen is in a concentration range of 0.1-100 mg/ml with respect to the bio-ink formulation. In one another embodiment of the present disclosure, the modified hyaluronic acid is in the concentration range of 5-90 mg/mL, or 10-80 mg/mL, or 15-80 mg/mL, or 20-70 mg/mL, or 25-70 mg/mL, or 30-60 mg/mL, or 30-55 mg/mL, 30-50 mg/mL, or 30-47 mg/mL with respect to the bio-ink formulation, and wherein the modified collagen peptide is in the concentration range of 20-230 mg/ml, or 50-200 mg/ml, or 75-200 mg/ml, or 90-200 mg/ml, or 100-200 mg/ml, or 125-175 mg/ml, and wherein the modified collagen is in a concentration range of 0.5-90 mg/ml, or 1-80 mg/ml, or 5-70 mg/ml, or 7-60 mg/ml, or 8-50 mg/ml, or 8-40 mg/ml, or 8-30 mg/ml, or 8-20 mg/ml.

In an embodiment of the present disclosure, there is provided a bio-ink formulation comprising: (a) a modified hyaluronic acid having a molecular weight in the range of 40-60 kDa, and with a degree of substitution in the range of 40-60%; (b) a modified collagen peptide having a molecular weight in the range of 40-60 kDa, and with a degree of substitution in the range of 40-60%; and (c) gelatin having a bloom value in the range of 175-225, wherein gelatin is in the concentration range of 50-70 mg/ml with respect to the bio-ink formulation, and wherein the modified hyaluronic acid is in a concentration range of 25-45 mg/ml, and wherein the modified collagen peptide is in a concentration range of 125-175 mg/ml, and wherein the modified hyaluronic acid is methacrylated hyaluronic acid, and the modified collagen peptide is thiolated collagen peptide.

In an embodiment of the present disclosure, there is provided a bio-ink formulation as described herein, wherein the bio-ink formulation further comprises a photo-activator, wherein the photo-activator is either eosin having a concentration in the range of 0.005-1 mM with respect to the bio-ink formulation, or the photo-activator is riboflavin having a concentration in the range of 0.1-50 mM with respect to the bio-ink formulation.

In an embodiment of the present disclosure, there is provided a bio-ink formulation as described herein, wherein the bio-ink formulation further comprises a photo-activator eosin having a concentration in the range of 0.005-1 mM with respect to the bio-ink formulation. In another embodiment of the present disclosure, eosin has a concentration in the range of 0.005-1 mM, or 0.01-1 mM, or 0.05-1 mM, or 0.1-1 mM, or 0.5-1 mM, or 0.75-1 mM with respect to the bio-ink formulation.

In an embodiment of the present disclosure, there is provided a bio-ink formulation as described herein, wherein the bio-ink formulation further comprises a photo-activator riboflavin having a concentration in the range of 0.1-50 mM with respect to the bio-ink formulation. In another embodiment of the present disclosure, riboflavin has a concentration in the range of 1-45 mM, or 5-40 mM, or 10-35 mM, or 15-30 mM, or 17-25 mM, with respect to the bio-ink formulation.

In an embodiment of the present disclosure, there is provided a bio-ink formulation as described herein, wherein the bio-ink formulation further comprises stem cells selected from the group consisting of human corneal stromal stem cells, human corneal limbal stem cells, human bone marrow-derived mesenchymal stem cells, adipose tissue-derived mesenchymal stem cells, umbilical cord-derived mesenchymal stem cells, Wharton jelly-derived mesenchymal stem cells, dental pulp derived mesenchymal stem cells, placental mesenchymal stem cells, and induced pluripotent stem cells. In another embodiment of the present disclosure, the stem cells are in the range of 0.1-100 million cells/ml of the bio-ink formulation. In yet another embodiment of the present disclosure, the stem cells are in the range of 1-100, or 10-100, or 20-90, or 30-80, or 40-90, or 50-100 million cells/ml of the bio-ink formulation.

In an embodiment of the present disclosure, there is provided a bio-ink formulation as described herein, wherein the bio-ink formulation further comprises exosomes selected from the group consisting of naive mesenchymal stem cell-derived exosomes, primed mesenchymal stem cell derived-exosomes, and corneal stromal stem cell derived-exosomes, and wherein the primed mesenchymal stem cell derived-exosomes are exosomes derived from corneal stromal stem cell derived-conditioned medium primed mesenchymal stem cells. In another embodiment of the present disclosure, the exosomes has a concentration in the range of 0.5-25 billion exosomes per ml of the bio-ink formulation. In yet another embodiment of the present disclosure, the exosomes has a concentration in the range of 1-20, or 3-20, or 5-20, or 10-25 billion per ml.

In an embodiment of the present disclosure, there is provided a bio-ink formulation as described herein, wherein the bio-ink formulation further comprises: (i) stem cells selected from the group consisting of human corneal stromal stem cells, human corneal limbal stem cells, human bone marrow-derived mesenchymal stem cells, adipose tissue-derived mesenchymal stem cells, umbilical cord-derived mesenchymal stem cells, Wharton jelly-derived mesenchymal stem cells, dental pulp derived mesenchymal stem cells, placental mesenchymal stem cells, and induced pluripotent stem cells; and (ii) exosomes selected from the group consisting of naive mesenchymal stem cell-derived exosomes, primed mesenchymal stem cell derived-exosomes, and corneal stromal stem cell derived-exosomes, and wherein the primed mesenchymal stem cell derived-exosomes are exosomes derived from corneal stromal stem cell derived-conditioned medium primed mesenchymal stem cells. In another embodiment of the present disclosure, the stem cells are in the range of 0.1-100. 1-100, or 10-100, or 20-90, or 30-80, or 40-90, or 50-100 million cells/ml of the bio-ink formulation, and wherein the exosomes has a concentration in the range of 0.5-25, 1-20, or 3-20, or 5-20, or 10-25 billion per ml.

In an embodiment of the present disclosure, there is provided a bio-ink formulation as described herein, wherein the bio-ink formulation has a viscosity in the range of 1690-5300 cP. In another embodiment of the present disclosure, the bio-ink formulation has a viscosity in the range of 1750-5200 cP, or 1800-5100 cP, or 1900-5000 cP, 2100-4800 cP, or 2300-5000 cP, or 2500-5300 cP, or 2000-5300 cP.

In an embodiment of the present disclosure, there is provided a process for preparing a bio-ink formulation as described herein, wherein the process comprises: (a) contacting a modified hyaluronic acid having a molecular weight in the range of 30-300 kDa, and with a degree of substitution in the range of 10-80%, and a modified collagen peptide having a molecular weight in the range of 10-80 kDa, and with a degree of substitution in the range of 10-80%; and gelatin having a bloom value in the range of 50-325, to obtain a first mixture; and (b) contacting the first mixture with a photo-activator to obtain the bio-ink formulation. In another embodiment of the present disclosure, contacting the modified hyaluronic acid, the modified collagen peptide, and gelatin is done at a temperature in the range of 33-38° C., for a time period in the range of 30-300 minutes, under dark condition to obtain the first mixture. In yet another embodiment of the present disclosure, the contacting is done at a temperature in the range of 34-38° C., or 35-38° C., or 36-38° C., and wherein the time period is in the range of 40-280, or 50-250, or 75-225, or 100-200 minutes.

In an embodiment of the present disclosure, there is provided a process for preparing a bio-ink formulation as described herein, wherein the process comprises: (a) contacting a modified hyaluronic acid having a molecular weight in the range of 30-300 kDa and with a degree of substitution in the range of 10-80%, and a modified collagen having a molecular weight in the range of 200-300 kDa and with a degree of substitution in the range of 10-80%, and gelatin having a bloom value in the range of 50-325, to obtain a first mixture; and (b) contacting the first mixture with a photo-activator to obtain the bio-ink formulation. In another embodiment of the present disclosure, contacting the modified hyaluronic acid, the modified collagen, and gelatin is done at a temperature in the range of 33-38° C., for a time period in the range of 30-300 minutes, under dark condition to obtain the first mixture. In yet another embodiment of the present disclosure, the contacting is done at a temperature in the range of 34-38° C., or 35-38° C., or 36-38° C., and wherein the time period is in the range of 40-280, or 50-250, or 75-225, or 100-200 minutes.

In an embodiment of the present disclosure, there is provided a process for preparing a bio-ink formulation as described herein, wherein the process comprises: (a) contacting a first polymer selected from the group consisting of modified hyaluronic acid, modified polyethylene glycol, modified polyvinyl alcohol, modified poly(N-isopropylacrylamide), modified alginate, silk, and modified silk, and a second polymer selected from the group consisting of collagen peptide, modified collagen peptide, collagen, and modified collagen, and a thickener selected from the group consisting of gelatin, modified cellulose, gellan gum, xanthum gum, polyethylene glycol, poloxamer, polyvinyl alcohol, and alginate, to obtain a first mixture; and (b) contacting the first mixture with a photo-activator to obtain the bio-ink formulation, wherein the bio-ink formulation is having a viscosity in the range of 1690-5300 cP. In another embodiment of the present disclosure, contacting the first polymer, the second polymer, and the thickener is done at a temperature in the range of 33-38° C., for a time period in the range of 30-300 minutes, under dark condition to obtain the first mixture. In yet another embodiment of the present disclosure, the contacting is done at a temperature in the range of 34-38° C., or 35-38° C., or 36-38° C., and wherein the time period is in the range of 40-280, or 50-250, or 75-225, or 100-200 minutes.

In an embodiment of the present disclosure, there is provided a bio-printed corneal lenticule comprising the bio-ink formulation as described herein.

In an embodiment of the present disclosure, there is provided a bio-printed corneal lenticule comprising a bio-ink formulation, said formulation comprising: (a) a modified hyaluronic acid having a molecular weight in the range of 30-300 kDa, and with a degree of substitution in the range of 10-80%, and having a weight percentage in the range of 3.1-5% with respect to the bio-printed corneal lenticule; (b) a modified collagen peptide having a molecular weight in the range of 10-80 kDa, and with a degree of substitution in the range of 10-80%, and having a weight percentage in the range of 8-20% with respect to the bio-printed corneal lenticule; and (c) gelatin having a bloom value in the range of 50-325, wherein gelatin is in the concentration range of 0.01-15% with respect to the bio-printed corneal lenticule, preferably in the range of 5-10% with respect to the bio-printed corneal lenticule .

In an embodiment of the present disclosure, there is provided a bio-printed corneal lenticule comprising a bio-ink formulation, said formulation comprising: (a) a modified hyaluronic acid having a molecular weight in the range of 30-300 kDa, and with a degree of substitution in the range of 10-75%, and having a weight percentage in the range of 3.1-5% with respect to the bio-printed corneal lenticule; (b) a modified collagen having a molecular weight in the range of 200-300 kDa, and with a degree of substitution in the range of 10-75%, and having a weight percentage in the range of 8-20% with respect to the bio-printed corneal lenticule; and (c) gelatin having a bloom value in the range of 50-325, wherein gelatin is in the concentration range of 0.01-15% with respect to the bio-printed corneal lenticule, preferably in the range of 5-10% with respect to the bio-printed corneal lenticule.

In an embodiment of the present disclosure, there is provided a process for obtaining a bio-printed corneal lenticule, said process comprising: (a) obtaining a bio-ink formulation as described herein; (b) printing the bio-ink formulation over a scaffold to obtain a printed corneal structure; and (c) exposing the printed corneal structure to a light having a wavelength in the range of 420-570 nm, and having an intensity in the range of 50-150 mW/cm² for a time period in the range of 1-15 minutes for obtaining the bio-printed corneal lenticule. In another embodiment of the present disclosure, the intensity of the light is in the range of 75-150, or 80-140, or 90-140, or 95-130 mW/cm², and wherein the time period in the range of 2-12, or 4-10, or 5-15 minutes.

In an embodiment of the present disclosure, there is provided a process for obtaining a bio-printed corneal lenticule, said process comprising: (i) obtaining a bio-ink formulation comprising: (a) a modified hyaluronic acid having a molecular weight in the range of 30-300 kDa, and with a degree of substitution in the range of 10-80%; (b) a modified collagen peptide having a molecular weight in the range of 10-80 kDa, and with a degree of substitution in the range of 10-80%; and (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation; (ii) printing the bio-ink formulation over a scaffold to obtain a printed corneal structure; and (iii) exposing the printed corneal structure to a light having a wavelength in the range of 420-570 nm, and having an intensity in the range of 50-150 mW/cm²for a time period in the range of 1-15 minutes for obtaining the bio-printed corneal lenticule. In another embodiment of the present disclosure, the intensity of the light is in the range of 75-150, or 80-140, or 90-140, or 95-130 mW/cm², and wherein the time period in the range of 2-12, or 4-10, or 5-15 minutes.

In an embodiment of the present disclosure, there is provided a process for obtaining a bio-printed corneal lenticule, said process comprising: (i) obtaining a bio-ink formulation comprising: (a) (a) a modified hyaluronic acid having a molecular weight in the range of 30-300 kDa, and with a degree of substitution in the range of 10-75%; (b) a modified collagen having a molecular weight in the range of 200-300 kDa, and with a degree of substitution in the range of 10-75%; and (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation; (ii) printing the bio-ink formulation over a scaffold to obtain a printed corneal structure; and (iii) exposing the printed corneal structure to a light having a wavelength in the range of 420-570 nm, and having an intensity in the range of 50-150 mW/cm²for a time period in the range of 1-15 minutes for obtaining the bio-printed corneal lenticule. In another embodiment of the present disclosure, the intensity of the light is in the range of 75-150, or 80-140, or 90-140, or 95-130 mW/cm², and wherein the time period in the range of 2-12, or 4-10, or 5-15 minutes.

In an embodiment of the present disclosure, there is provided a process for obtaining a bio-printed corneal lenticule, said process comprising: (i) obtaining a bio-ink formulation comprising: (a) a first polymer selected from the group consisting of modified hyaluronic acid, modified polyethylene glycol, modified polyvinyl alcohol, modified poly(N-isopropylacrylamide), modified alginate, silk, and modified silk; (b) a second polymer selected from the group consisting of collagen peptide, modified collagen peptide, collagen, and modified collagen; (c) a thickener selected from the group consisting of gelatin, modified cellulose, gellan gum, xanthum gum, polyethylene glycol, poloxamer, polyvinyl alcohol, and alginate, wherein the bio-ink formulation is having a viscosity in the range of 1690-5300 cP; (ii) printing the bio-ink formulation over a scaffold to obtain a printed corneal structure; and (iii) exposing the printed corneal structure to a light having a wavelength in the range of 420-570 nm, and having an intensity in the range of 50-150 mW/cm² for a time period in the range of 1-15 minutes for obtaining the bio-printed corneal lenticule. In another embodiment, the intensity of the light is in the range of 75-150, or 80-140, or 90-140, or 95-130 mW/cm², and wherein the time period in the range of 2-12, or 4-10, or 5-15 minutes.

In an embodiment of the present disclosure, there is provided a process for obtaining a bio-printed corneal lenticule, said process comprising: (a) obtaining a bio-ink formulation as described herein; (b) printing the bio-ink formulation over a scaffold to obtain a printed corneal structure; and (c) exposing the printed corneal structure to a light having a wavelength in the range of 420-570 nm, and having an intensity in the range of 50-150 mW/cm² for a time period in the range of 1-15 minutes for obtaining the bio-printed corneal lenticule, wherein the printing is done using a 3D printer.

In an embodiment of the present disclosure, there is provided a process for obtaining a bio-printed corneal lenticule, said process as described herein, wherein printing the first mixture over the scaffold is done at a temperature in the range of 22-30° C., at an extrusion pressure in the range of 5-80 kPa, and at a speed in the range of 1-20 mm/sec. In another embodiment of the present disclosure, printing the first mixture over the scaffold is done at a temperature in the range of 22-29° C., or 22-28 ° C., or 22-27 ° C., or 22-26 ° C., or 22-25 ° C., or 22.2-27 ° C., and wherein the speed is in the range of 2-18, or 5-16, or 7-12 mm/sec.

In an embodiment of the present disclosure, there is provided a bio-printed corneal lenticule obtained by a process comprising: (a) obtaining a bio-ink formulation as described herein; (b) printing the bio-ink formulation over a scaffold to obtain a printed corneal structure; and (c) exposing the printed corneal structure to a light having a wavelength in the range of 420-570 nm, and having an intensity in the range of 50-150 mW/cm²for a time period in the range of 1-15 minutes for obtaining the bio-printed corneal lenticule. In another embodiment of the present disclosure, the intensity of the light is in the range of 75-150, or 80-140, or 90-140, or 95-130 mW/cm², and wherein the time period in the range of 2-12, or 4-10, or 5-15 minutes.

In an embodiment of the present disclosure, there is provided a bio-printed corneal lenticule obtained by a process comprising: (i) obtaining a bio-ink formulation comprising: (a) a modified hyaluronic acid having a molecular weight in the range of 30-300 kDa, and with a degree of substitution in the range of 10-80%; (b) a modified collagen peptide having a molecular weight in the range of 10-80 kDa, and with a degree of substitution in the range of 10-80%; and (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation; (ii) printing the bio-ink formulation over a scaffold to obtain a printed corneal structure; and (iii) exposing the printed corneal structure to a light having a wavelength in the range of 420-570 nm, and having an intensity in the range of 50-150 mW/cm² for a time period in the range of 1-15 minutes for obtaining the bio-printed corneal lenticule. In another embodiment of the present disclosure, the intensity of the light is in the range of 75-150, or 80-140, or 90-140, or 95-130 mW/cm², and wherein the time period in the range of 2-12, or 4-10, or 5-15 minutes.

In an embodiment of the present disclosure, there is provided a bio-printed corneal lenticule obtained by a process as described herein, wherein gelatin having a weight percentage of 60-65% is leached out from the bio-printed corneal lenticule over a time period of 20-25 hours under in-vitro conditions. In another embodiment, the 60-64% gelatin is leached out over a time period in a range of 20-24 hours. In yet another embodiment, the in-vitro conditions refer to a suitable medium in which the bio-printed corneal lenticule is stored. The in-vitro conditions can also be a suitable culture medium.

In an embodiment of the present disclosure, there is provided a bio-printed corneal lenticule obtained by a process comprising: (i) obtaining a bio-ink formulation comprising: (a) a modified hyaluronic acid having a molecular weight in the range of 30-300 kDa, and with a degree of substitution in the range of 10-75%; (b) a modified collagen having a molecular weight in the range of 200-300 kDa, and with a degree of substitution in the range of 10-75%; and (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation; (ii) printing the bio-ink formulation over a scaffold to obtain a printed corneal structure; and (iii) exposing the printed corneal structure to a light having a wavelength in the range of 420-570 nm, and having an intensity in the range of 50-150 mW/cm² for a time period in the range of 1-15 minutes for obtaining the bio-printed corneal lenticule. In another embodiment of the present disclosure, the intensity of the light is in the range of 75-150, or 80-140, or 90-140, or 95-130 mW/cm², and wherein the time period in the range of 2-12, or 4-10, or 5-15 minutes.

In an embodiment of the present disclosure, there is provided a bio-printed corneal lenticule obtained by a process comprising: (i) obtaining a bio-ink formulation comprising: (a) a first polymer selected from the group consisting of modified hyaluronic acid, modified polyethylene glycol, modified polyvinyl alcohol, modified poly(N-isopropylacrylamide), modified alginate, silk, and modified silk; (b) a second polymer selected from the group consisting of collagen peptide, modified collagen peptide, collagen, and modified collagen; (c) a thickener selected from the group consisting of gelatin, modified cellulose, gellan gum, xanthum gum, polyethylene glycol, poloxamer, polyvinyl alcohol, and alginate, wherein the bio-ink formulation is having a viscosity in the range of 1690-5300 cP; (ii) printing the bio-ink formulation over a scaffold to obtain a printed corneal structure; and (iii) exposing the printed corneal structure to a light having a wavelength in the range of 420-570 nm, and having an intensity in the range of 50-150 mW/cm² for a time period in the range of 1-15 minutes for obtaining the bio-printed corneal lenticule. In another embodiment of the present disclosure, the intensity of the light is in the range of 75-150, or 80-140, or 90-140, or 95-130 mW/cm², and wherein the time period in the range of 2-12, or 4-10, or 5-15 minutes.

In an embodiment of the present disclosure, there is provided a bio-printed corneal lenticule obtained by a process comprising: (i) obtaining a bio-ink formulation comprising: (a) a modified hyaluronic acid having a molecular weight in the range of 30-300 kDa, and with a degree of substitution in the range of 10-80%; (b) a modified collagen peptide having a molecular weight in the range of 10-80 kDa, and with a degree of substitution in the range of 10-80%; (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation; and (d) stem cells selected from the group consisting of human corneal stromal stem cells, human corneal limbal stem cells, human bone marrow-derived mesenchymal stem cells, adipose tissue-derived mesenchymal stem cells, umbilical cord-derived mesenchymal stem cells, Wharton jelly-derived mesenchymal stem cells, dental pulp derived mesenchymal stem cells, placental mesenchymal stem cells, and induced pluripotent stem cells; (ii) printing the bio-ink formulation over a scaffold to obtain a printed corneal structure; and (iii) exposing the printed corneal structure to a light having a wavelength in the range of 420-570 nm, and having an intensity in the range of 50-150 mW/cm² for a time period in the range of 1-15 minutes for obtaining the bio-printed corneal lenticule.

In an embodiment of the present disclosure, there is provided a bio-printed corneal lenticule obtained by a process comprising: (i) obtaining a bio-ink formulation comprising: (a) a modified hyaluronic acid having a molecular weight in the range of 30-300 kDa, and with a degree of substitution in the range of 10-80%; (b) a modified collagen peptide having a molecular weight in the range of 10-80 kDa, and with a degree of substitution in the range of 10-80%; (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation; and (d) exosomes selected from the group consisting of naive mesenchymal stem cell-derived exosomes, primed mesenchymal stem cell derived-exosomes, and corneal stromal stem cell derived-exosomes; (ii) printing the bio-ink formulation over a scaffold to obtain a printed corneal structure; and (iii) exposing the printed corneal structure to a light having a wavelength in the range of 420-570 nm, and having an intensity in the range of 50-150 mW/cm² for a time period in the range of 1-15 minutes for obtaining the bio-printed corneal lenticule.

In an embodiment of the present disclosure, there is provided a bio-printed corneal lenticule obtained by a process comprising: (i) obtaining a bio-ink formulation comprising: (a) a modified hyaluronic acid having a molecular weight in the range of 30-300 kDa, and with a degree of substitution in the range of 10-80%; (b) a modified collagen peptide having a molecular weight in the range of 10-80 kDa, and with a degree of substitution in the range of 10-80%; (c) gelatin having a bloom value in the range of 50-325, wherein gelatin is in the concentration range of 0.1-150 mg/ml with respect to the bio-ink formulation, preferably in the range of 50-100 mg/ml with respect to the bio-ink formulation; (d) exosomes selected from the group consisting of naive mesenchymal stem cell-derived exosomes, primed mesenchymal stem cell derived-exosomes, and corneal stromal stem cell derived-exosomes; and (e) exosomes selected from the group consisting of naive mesenchymal stem cell-derived exosomes, primed mesenchymal stem cell derived-exosomes, and corneal stromal stem cell derived-exosomes; (ii) printing the bio-ink formulation over a scaffold to obtain a printed corneal structure; and (iii) exposing the printed corneal structure to a light having a wavelength in the range of 420-570 nm, and having an intensity in the range of 50-150 mW/cm² for a time period in the range of 1-15 minutes for obtaining the bio-printed corneal lenticule.

In an embodiment of the present disclosure, there is provided a method for treating a corneal defect in a subject, said method comprises: (a) obtaining the bio-printed corneal lenticule as described herein; and (b) implanting the bio-printed corneal lenticule at the site of the corneal defect, for treating the corneal defect in the subject.

In an embodiment of the present disclosure, there is provided a method for treating a corneal defect in a subject, said method comprises: (a) obtaining the bio-printed corneal lenticule as described herein; and (b) implanting the bio-printed corneal lenticule at the site of the corneal defect, for treating the corneal defect in the subject, wherein the subject is administered with a pharmaceutically acceptable amount of a formulation comprising: (i) exosomes selected from the group consisting of corneal stromal stem cell derived-exosomes, primed mesenchymal stem cell derived-exosomes, and naive mesenchymal stem cell derived-exosomes; and (ii) a clinically approved eye drop formulation, and wherein the administration is done before or after implanting the bio-printed corneal lenticule.

In an embodiment of the present disclosure, there is provided a method for treating a corneal defect in a subject, said method comprises: (a) obtaining the bio-printed corneal lenticule as described herein; and (b) implanting the bio-printed corneal lenticule at the site of the corneal defect, for treating the corneal defect in the subject, wherein the subject is administered with a pharmaceutically acceptable amount of a formulation comprising: (i) exosomes selected from the group consisting of corneal stromal stem cell derived-exosomes, primed mesenchymal stem cell derived-exosomes, and naive mesenchymal stem cell derived-exosomes; and (ii) a clinically approved eye drop formulation, and wherein the administration is done before or after implanting the bio-printed corneal lenticule, and wherein the exosomes is selected from the group consisting of naive mesenchymal stem cell-derived exosomes, primed mesenchymal stem cell derived-exosomes, and corneal stromal stem cell derived-exosomes, and wherein the exosomes has a concentration in the range of 0.5-25 billion exosomes per ml of the formulation, and wherein the clinically approved formulation comprises at least one polymer selected from the group consisting of hyaluronic acid, carboxymethyl cellulose, polyethylene glycol, polyvinyl alcohol, propylene glycol, and alginate.

In an embodiment of the present disclosure, there is provided a bio-printed corneal lenticule as described herein, wherein the bio-printed corneal lenticule has a thickness in the range of 10-500 microns. In another embodiment of the present disclosure, the thickness is in the range of 20-490, or 50-500, or 50-450, or 75-400, or 100-500, or 100-400, or 200-400, or 250-500 microns.

In an embodiment of the present disclosure, there is provided a bio-printed corneal lenticule as described herein, wherein gelatin is gradually leached out from the lenticule. In another embodiment, 60-65% gelatin is leached out over a time period of 22-24 hours.

In an embodiment of the present disclosure, there is provided a bio-printed corneal lenticule as described herein, wherein the bio-printed corneal lenticule has a transmittance to a visible light of 350-750 nm, in the range of 80-99%. In another embodiment of the present disclosure, the transmittance is in the range of 82-99%, or 84-99%, or 86-99%, or 88-99%, or 90-99%, or 92-99%, or 94-99%.

In an embodiment of the present disclosure, there is provided a bio-printed corneal lenticule as described herein, wherein the bio-printed corneal lenticule has a degradation percentage under suitable conditions in the range of 2-40% within 30 days.

In an embodiment of the present disclosure, there is provided a bio-printed corneal lenticule as described herein, wherein the bio-printed corneal lenticule has a compressive modulus in the range of 100-650 kPa. In another embodiment of the present disclosure, the compressive modulus is in the range of 150-650, or 200-650, or 250-650, or 300-650, or 350-650, or 400-650 kPa.

In an embodiment of the present disclosure, there is provided a bio-printed corneal lenticule as described herein, wherein the bio-printed corneal lenticule has a tensile strength in the range of 2-50 kPa.

In an embodiment of the present disclosure, there is provided a bio-printed corneal lenticule as described herein for use in treating a corneal defect in a subject.

In an embodiment of the present disclosure, there is provided a bio-printed corneal lenticule as described herein for use in in-vitro studies for testing drug toxicity, and disease modelling.

In an embodiment of the present disclosure, there is provided a bio-ink formulation as described herein for use in preparing a bio-printed corneal lenticule.

In an embodiment of the present disclosure, there is provided a bio-ink formulation as described herein, wherein the bio-ink formulation further comprises at least one component from decellularized extracellular matrix.

In an embodiment of the present disclosure, there is provided a bio-ink formulation as described herein, wherein the bio-ink formulation further comprises at least one cell-derived component.

In an embodiment of the present disclosure, there is provided a bio-printed corneal lenticule comprising: (a) a modified hyaluronic acid having a molecular weight in the range of 30-300 kDa, and with a degree of substitution in the range of 10-80%, and having a weight percentage in the range of 0.2-10% with respect to the bio-printed corneal lenticule; (b) a modified collagen peptide having a molecular weight in the range of 10-80 kDa, and with a degree of substitution in the range of 10-80%, and having a weight percentage in the range of 1-25% with respect to the bio-printed corneal lenticule; and (c) gelatin having a bloom value in the range of 50-325, and having a weight percentage in the range of 0.01-15% with respect to the bio-printed corneal lenticule. In another embodiment of the present disclosure, the modified hyaluronic acid has a molecular weight in the range of 35-250, or 35-200 kDa, or 40-175 kDa, or 40-150 kDa, or 40-125 kDa, or 40-100 kDa, or 40-75 kDa, and with a degree of substitution in the range of 20-80%, or 25-75%, or 30-70%, or 35-65%, or 40-60%, and having a weight percentage in the range of 0.5-10%, or 1-8%, or 2-6%, or 2.5-5% with respect to the bio-printed corneal lenticule. In yet another embodiment of the present disclosure, the modified collagen peptide having a molecular weight in the range of 20-75 kDa, or 25-70 kDa, or 30-65 kDa, or 35-60 kDa, or 40-60 kDa, and with a degree of substitution in the range of 20-70%, or 30-60%, or 35-60%, or 40-60%, and having a weight percentage in the range of 5-25%, or 10-25%, or 10-20% with respect to the bio-printed corneal lenticule. In an alternate embodiment of the present disclosure, gelatin has a weight percentage in the range of 0.05-15%, or 2-15%, or 5-15%, or 5-10%.

In an embodiment of the present disclosure, there is provided a bio-printed corneal lenticule comprising: (a) a modified hyaluronic acid having a molecular weight in the range of 30-300 kDa, and with a degree of substitution in the range of 10-80%, and having a weight percentage in the range of 0.2-10% with respect to the bio-printed corneal lenticule; (b) a modified collagen having a molecular weight in the range of 200-300 kDa, and with a degree of substitution in the range of 10-75%, and having a weight percentage in the range of 5-50% with respect to the bio-printed corneal lenticule; and (c) gelatin having a bloom value in the range of 50-325, and having a weight percentage in the range of 0.01-15% with respect to the bio-printed corneal lenticule.

In an embodiment of the present disclosure, there is provided a bio-printed corneal lenticule comprising: (a) a modified hyaluronic acid having a molecular weight in the range of 30-300 kDa, and with a degree of substitution in the range of 10-80%, and having a weight percentage in the range of 0.2-10% with respect to the bio-printed corneal lenticule; (b) a modified collagen peptide having a molecular weight in the range of 10-80 kDa, and with a degree of substitution in the range of 10-80%, and having a weight percentage in the range of 1-25% with respect to the bio-printed corneal lenticule; (c) gelatin having a bloom value in the range of 50-325, and having a weight percentage in the range of 0.01-15% with respect to the bio-printed corneal lenticule; and (d) stem cells selected from the group consisting of human corneal stromal stem cells, human corneal limbal stem cells, human bone marrow-derived mesenchymal stem cells, adipose tissue-derived mesenchymal stem cells, umbilical cord-derived mesenchymal stem cells, Wharton jelly-derived mesenchymal stem cells, dental pulp derived mesenchymal stem cells, placental mesenchymal stem cells, and induced pluripotent stem cells.

In an embodiment of the present disclosure, there is provided a bio-printed corneal lenticule comprising: (a) a modified hyaluronic acid having a molecular weight in the range of 30-300 kDa, and with a degree of substitution in the range of 10-80%, and having a weight percentage in the range of 0.2-10% with respect to the bio-printed corneal lenticule; (b) a modified collagen peptide having a molecular weight in the range of 10-80 kDa, and with a degree of substitution in the range of 10-80%, and having a weight percentage in the range of 1-25% with respect to the bio-printed corneal lenticule; (c) gelatin having a bloom value in the range of 50-325, and having a weight percentage in the range of 0.01-15% with respect to the bio-printed corneal lenticule; (d) stem cells selected from the group consisting of human corneal stromal stem cells, human corneal limbal stem cells, human bone marrow-derived mesenchymal stem cells, adipose tissue-derived mesenchymal stem cells, umbilical cord-derived mesenchymal stem cells, Wharton jelly-derived mesenchymal stem cells, dental pulp derived mesenchymal stem cells, placental mesenchymal stem cells, and induced pluripotent stem cells; and (e) exosomes selected from the group consisting of naive mesenchymal stem cell-derived exosomes, primed mesenchymal stem cell derived-exosomes, and corneal stromal stem cell derived-exosomes.

In an embodiment of the present disclosure, there is provided a bio-printed corneal lenticule comprising: (a) a modified hyaluronic acid having a molecular weight in the range of 30-300 kDa, and with a degree of substitution in the range of 10-80%, and having a weight percentage in the range of 0.2-10% with respect to the bio-printed corneal lenticule; (b) a modified collagen peptide having a molecular weight in the range of 10-80 kDa, and with a degree of substitution in the range of 10-80%, and having a weight percentage in the range of 1-25% with respect to the bio-printed corneal lenticule; (c) gelatin having a bloom value in the range of 50-325, and having a weight percentage in the range of 0.01-15% with respect to the bio-printed corneal lenticule; and (d) exosomes selected from the group consisting of naive mesenchymal stem cell-derived exosomes, primed mesenchymal stem cell derived-exosomes, and corneal stromal stem cell derived-exosomes.

### EXAMPLES

The disclosure will now be illustrated with a working example, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

### Example 1

### Materials Used in the Present Disclosure

The polymers and other materials used in the present disclosure were procured commercially. Table 1 depicts the commercial sources of the materials.

TABLE 1

| | | | | Source/ | Source/ |
|---|---|---|---|---|---|
| Constit- | | | | vendor | vendor |
| uents | Description | Sterilization | Form | (R&D) | (GMP) |
| 1 | HA-MA | EtO | lyophilized | Creative | — |
| | (2-100 | | | PEGWorks | |
| | mg/mL) | | | | |
| 2 | RCP-SH | EtO | lyophilized | Fujifilm | Fujifilm |
| | (10-250 | | | | |
| | mg/mL) | | | | |
| 3 | Eosin Y + | 0.22 um | lyophilized | Sigma | Sigma |
| | TEOA | membrane | | | |
| | | filter | | | |
| 4 | Cells | — | — | In-house, | CCT USA/ |
| | | | | USA | RoosterBio, |
| | | | | | USA/ |
| | | | | | Evercyte |
| | | | | | Gmbh, |
| | | | | | Austria |

| | | |
|---|---|---|
| 5 | Saline | Medical grade approved by Clinician |
| | | for reconstitution |

Methacrylated hyaluronic acid (HA-MA) was obtained from CreativePEG Works, and the thiolated recombinant collagen peptide (RCP-SH) was obtained from Fujifilm. Although, a non-limiting list of ingredients have been mentioned herein, however, a person skilled in the art can use any similar ingredient from any commercial source for the purpose of the present disclosure.

The Molecular weight of the components is based on the certificate of analysis provided by the commercial vendor. The HA-MA of 33 kDa as per the information from vendor has the degree of substitution of approximately 50%, and has a purity of 95%. The HA-MA of 50 kDa as per the information from vendor has the degree of substitution of approximately 47%, and has a purity of 95%.

The cells that can be used as a part of the bio-printed lenticule or can be used for culturing to obtain conditioned medium, and exosomes are covered below.

### Source of Primary Adult Stem Cells

Human corneal stromal stem cells (in-house), Bone marrow mesenchymal stem cells (BM-MSC) from Rooster Bio Inc. Human corneal epithelial cells, adipose-derived, umbilical cord-derived dental pulp-derived and Wharton Jelly-derived MSCs from Evercyte GmbH.

### Source of Immortalized Adult Stem Cells

1. Telomerized human Bone marrow derived mesenchymal stem cell line (BM-MSC/TERT277) was developed from mesenchymal stem cells isolated from spongy bone (sternum) by non-viral gene transfer of a plasmid carrying the hTERT gene. Positively transfected cells were selected by using neomycin phosphotransferase as selectable marker and Geneticin sulfate addition. The cell line was continuously cultured for more than 25 population doublings without showing signs of growth retardation or replicative senescence.
2. Telomerized human Wharton's Jelly derived mesenchymal stem cell line (WJ-MSC/TERT273) was established under xeno-free conditions from primary tissue disaggregation to non-viral transfer of hTERT.

The cell lines are characterized by unlimited growth while maintaining expression of cell type specific markers and functions such as:
Typical mesenchymal morphology
Expression of typical mesenchymal stem cell markers such as CD73, CD90 and CD105
Differentiation potential towards adipocytes, chondrocytes, osteoblasts
Production of extracellular vesicles with angiogenic and anti-inflammatory activity.

The biological cells mentioned above are only a few possible embodiments of the present disclosure, however, this is a non-limiting list and any other cell which suits the requirement can be used as a part of the present disclosure.

The molecular weights, and degree of substitution mentioned in the present disclosure in this section are the details mentioned in the certificate of analysis for the respective ingredient as provided by the vendor. For example, “33 kDa” of HA-MA refers to the HA-MA polymer of 33 kDa molecular weight as provided by the vendor, similar is the case with degree of substitution for example 50% DoS refers to the 50% degree of substitution as provided by the vendor.

### Example 2

### Bio-Ink Formulations, and Preparations Thereof

The present example describes the strategy employed for obtaining a bio-ink formulation which is amenable for serving as an ink in a 3D printer, and which also gives desirable characteristics of the bio-ink and the bio-printed corneal lenticule.

For bioprinting a polymer solution (bio-ink formulation) using nozzle of definite diameter, the ink (bio-ink formulation) should be viscous enough to support the printing parameters to obtain the required product (bio-printed corneal lenticule). The mixing of the two major components, methacrylated hyaluronic acid (HA-MA) and thiolated recombinant collagen peptide (RCP-SH) in different combinations of molecular weight, and concentrations, was assessed to obtain a desirable bio-ink having sufficient viscosity in the range of 1690 cP to 5300 cP.

### Preparation of the Bio-Ink Formulation

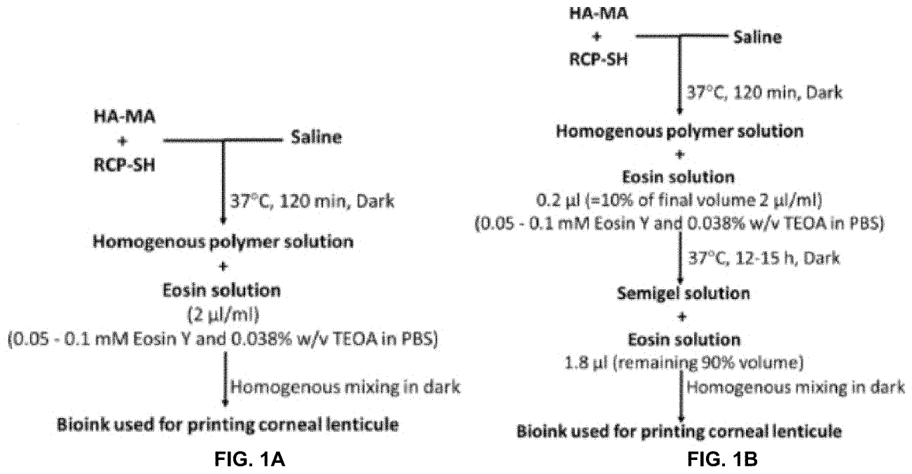

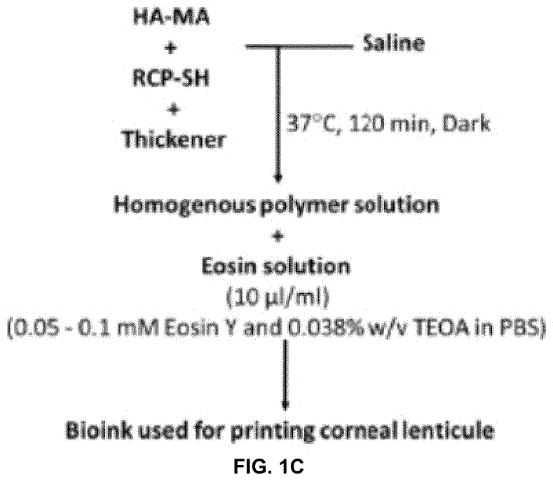

The functionalized hyaluronic acid (HA) can be mixed with functionalized RCP in the required concentration ratio and dissolved to obtain a homogenous solution in saline. In the first approach, the photo-initiator can be mixed with the polymer mixture in a single dose (FIG. 1A) just before the bioprinting process, which results in a low viscous solution that limits the printability of the bio-ink. In the second approach, the photo-initiator (eosin solution) can be added to the polymers in 2 steps. Here 10% of the eosin volume is first added and incubated with the polymer mix overnight to yield a semi-gel solution. This semi-gel solution is of higher viscosity, which greatly enhances the printability of the bio-ink. The remaining photo-initiator volume is added just before the bioprinting process (FIG. 1B). This approach enhances the range of printability for a given concentration range, which would otherwise have less viscosity for bioprinting, and restores the physical and biological properties of the bio-printed lenticule. However, inconsistent results were observed with the semi-gel approach as they were unstable after small periods of time, leading to the pre-gel solution highly prone to gelling inside the printing cartridge. Thus, a third method was assessed whereby a thickener was used to increase the overall viscosity of the polymer solutions to bring it into printable range, and which could be easily leached out after printing (FIG. 1C). Depending on the type of thickeners, its removal can be externally controlled by modulating temperature, washing or by using mild chemicals. In effect, the printability of the polymer solution (bio-ink formulation) can be easily controlled by changing the concentration of the thickener, without the need of increasing the biopolymer concentration that would have made the matrix stiff and difficult for cells to grow, and its removal after printing restores the expected matrix characteristics.

### Molecular Weights of HA-MA and RCP-SH in the Bio-Ink Formulation

In order to ensure that the bio-ink so developed possessed the properties essential for bioprinting, a series of experiments were performed based on following variables: Molecular weight of HA-MA (33 kDa to 250 kDa), degree of substitution (DoS) with the methacrylate group (30% or 50%) and combination with thiolated recombinant collagen peptide (RCP-SH) in different concentration.

The bioprinter that was employed for the experiment was Cellink BioX® which has a pressure range of 0-200 kPa and can print bio-inks with viscosity as high as ˜100,000 cP (=1000 Pa-s). In order to ensure that the bio-ink developed had the desired viscosity, two reference standards were chosen that had a wide difference in viscosity but were still printable, such as Cellink® test ink (65,000 cP) and Alginate (2%)+gelatin (4%) formulation (2126 cP).

Since the viscosity of a polymer solution is dependent on its molecular weight and concentration, HA-MA of different molecular weights (250 kDa, 50 kDa and 33 kDa) and degree of substitution (DoS) (30% and 50%) were analyzed. The range of viscosity assessed was decided on the basis of stability of the solution and ease of handling.

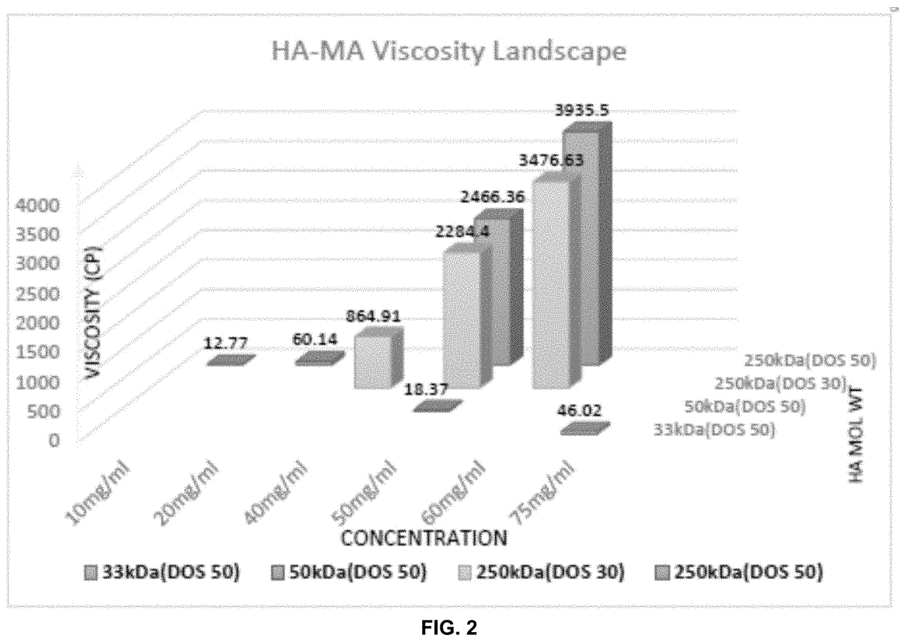

FIG. 2 depicts the results of the bio-ink formulation obtained by employing the method as described in the FIG. 1A. The results (FIG. 2) indicate that for bio-ink formulations with the same polymer concentration, viscosity is directly proportional to its molecular weight. The solutions prepared using 40-60 mg/ml of 250 kDa HA-MA showed viscosity values within the required range whereas, the degree of substitution had minimal effect on the viscosity.

### Concentrations of 250 kDa HA-MA (DoS 30%) or 50 kDa HA-MA (50% DoS) with RCP-SH (-51 kDa) on the Basis of Viscosity

The viscosity of the bio-ink formulations was assessed by varying the concentration of 250 kDa HA-MA (30% DoS) with 50 mg/ml RCP-SH (DoS 50%). To further study the effect of mode of photo-initiator (eosin) addition, 0.2 μL eosin was added to the bio-ink formulations and incubated overnight, followed by the addition of the remaining 1.8 μL eosin prior to the bioprinting process (as described in FIG. 1B). In the second approach, 2 μL eosin was added only prior to bioprinting (as described in FIG. 1C).

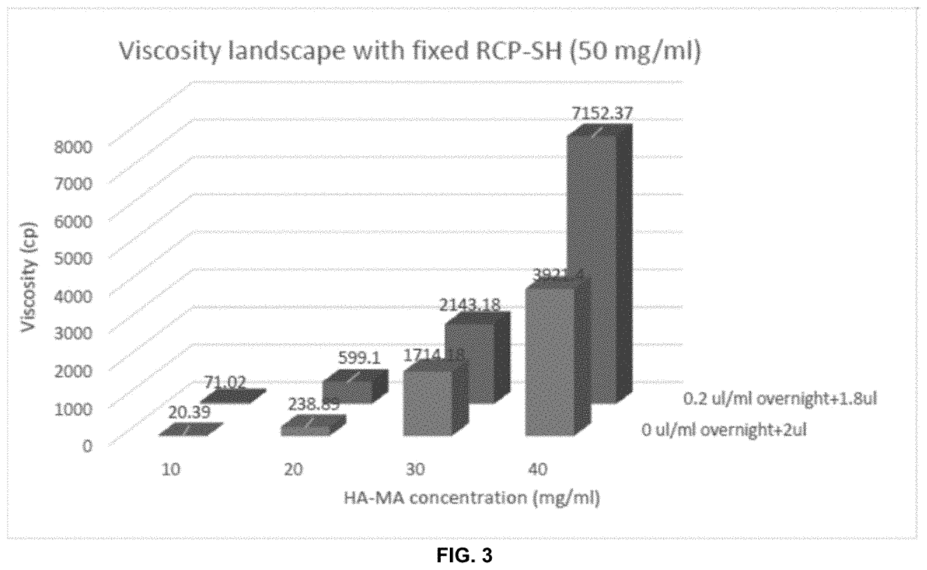

The results (FIG. 3) revealed that the overnight incubation of the polymer components significantly increased the viscosities of the solution (bio-ink obtained by the method disclosed in the FIG. 1B). The higher concentration of HA-MA (50 and 60 mg/mL) with 50 mg/ml RCP-SH solutions, were found to be unstable and gelled before the viscosity could be measured. Whereas, the solutions containing HA-MA in low concentrations (10 and 20 mg/mL) the viscosity was found to be correspondingly low, which in turn resulted in accumulation of the gel at the center of the mold during the printing and crosslinking processes. The solutions with 30 and 40 mg/mL HA-MA, with and without prior addition of eosin, yielded viscosities within the desired range.

The methods as described above did not yield a bio-ink formulation with all the desirable qualities, therefore, two more approaches were assessed. First, a semi-gel method where the components were mixed together and allowed to form a thicker solution (semi-gel) by incubating them together for longer time. This led to an increase in viscosity, but the pre-gel mix was very unstable and did not provide adequate time for bioprinting the solution. The second approach involved using a thickener which, apart from improving the viscosity, provided a thermo-responsive stability to the pre-gel solution.

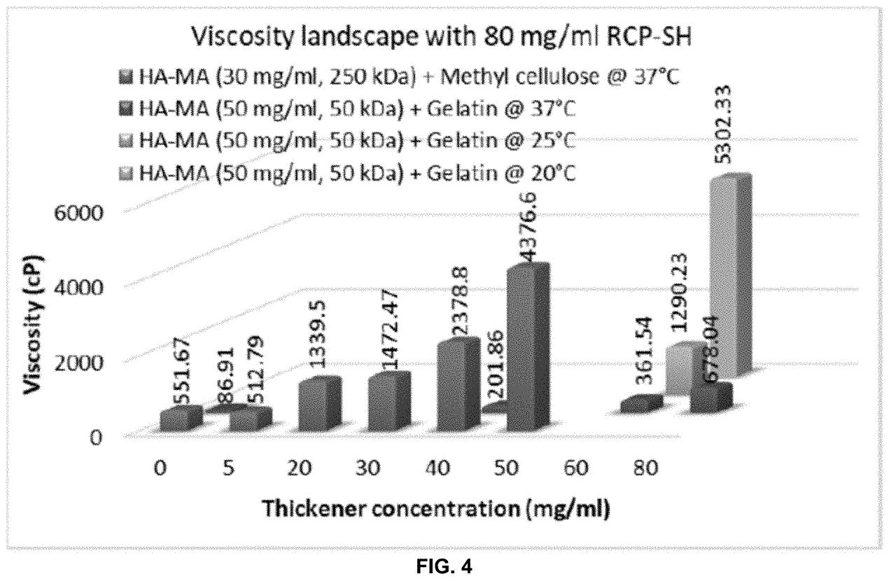

The result of viscosity evaluation on the addition of thickeners (FIG. 4) also revealed that viscosity is a function of thickener concentration. Keeping the concentration of HA-MA (250 kDa)/RCP-SH fixed at 30/80 (mg/ml) and assessing the effect of the addition of methyl cellulose (MC, 14 kDa) on the viscosity of the solution at 37° C. revealed that the concentration of MC had to be 20 mg/ml or more for increasing the viscosity of the solution by at least 2 times.

Whereas, when gelatin (medium bloom, 40-50 kDa) was used as a thickener for the 50 kDa HA-MA based system, it was observed that in addition to the concentration of gelatin, decreasing the temperature also had a significant effect on the viscosity of the bio-ink formulation. The bio-ink was prepared as per the method disclosed in the FIG. 1C. The gelatin used herein was of medium bloom having a bloom value in the range of 175-225, which translates into the range of 40-50 kDa.

### Example 3

### Printing of the Bio-Ink Formulations and the Effect of Thickener

### Process of Obtaining the Bio-Printed Corneal Lenticule Using the Bio-Ink Formulations

The bioprinter that was employed for the experiment was Cellink BioX® which has a pressure range of 0-200 kPa and can print bio-inks with viscosity as high as ˜100,000 cP (=1000 Pa-s). In order to ensure that the bio-ink developed had the desired viscosity, two reference standards were chosen that had a wide difference in viscosity but were still printable, such as Cellink® test ink (65,000 cP) and Alginate (2%)+gelatin (4%) formulation (2126 cP).

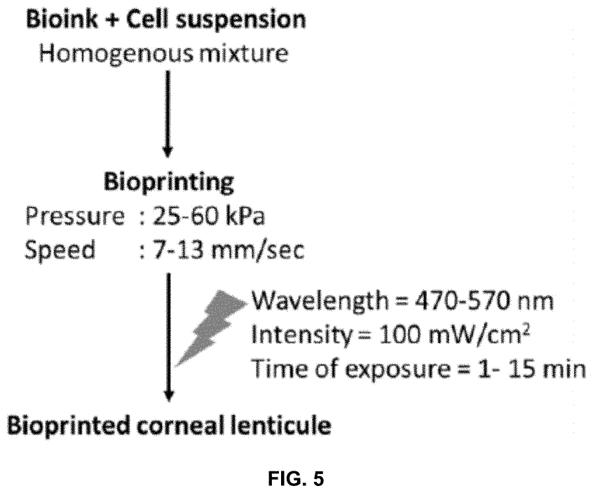

FIG. 5 depicts the process in a schematic manner. The bio-ink was transferred to the syringe (printhead) having 22G nozzle. In case where cells are required, the bio-ink along with the cells, are transferred to the syringe. Based on the inputs provided by the software for printing speed, pressure, shape etc., the print-head would move over the mold (scaffold) extruding the contents of the syringe. The printed structure was exposed to high-intensity light (100 mW/cm²) of wavelength 470-570 nm, for a total time of 4-5 min, to yield a bio-printed corneal lenticule. This can be removed from the mold and transferred to the culture medium for maintaining the encapsulated cells (in case the cells are used) in their required physiological state.

### Printability Assessment of the Bio-Ink Formulations

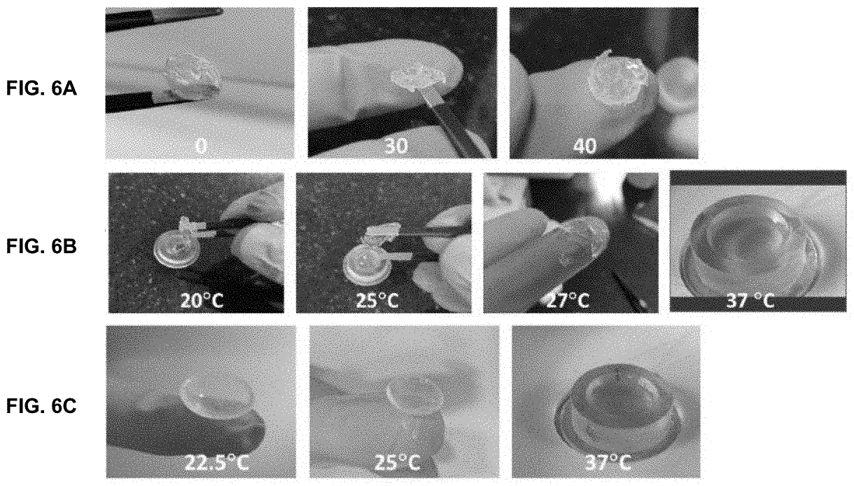

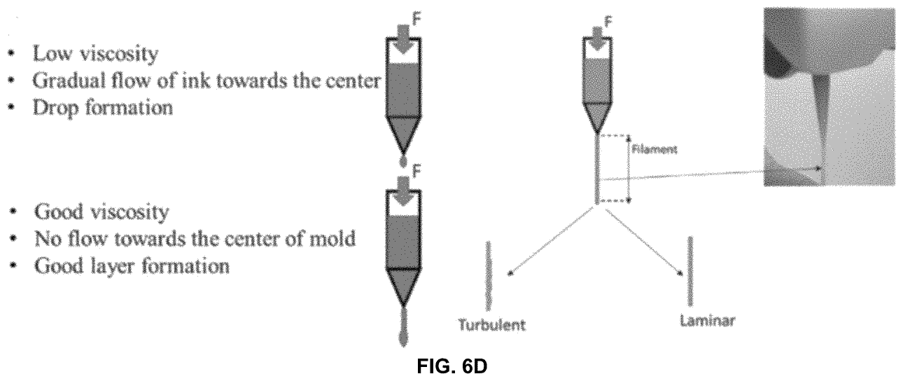

The thickeners were added to the biopolymer solutions in different concentrations, to obtain different bio-ink formulations, and their printability was assessed (Table 2 & 3, FIGS. 6A-6C). The lenticules printed using different concentration of MC with 30 mg/ml of 250 kDa HA-MA (30% DoS) and 80 mg/ml of 50 kDa RCP-SH (50% DoS) as the bio-ink resulted in soft and brittle lenticules which couldn't hold the shape and disintegrated on being removed from the molds (FIG. 6A). Further increasing the biopolymer concentration was not possible as the solution became too viscous and unstable on the addition of photo-initiator. Thus, lower molecular weight HA-MA was used, wherein the concentration range could be increased as per requirement. The lenticules printed using 60 mg/ml gelatin (medium bloom, 40-50 kDa) with 50 mg/ml of 50 kDa HA-MA (50% DoS) and 80 mg/ml of 50 kDa RCP-SH (50% DoS) combination as bio-ink provided good printability at different temperatures but were brittle (FIG. 6B).

Whereas, on changing the biopolymer combination to 35 mg/ml of 50 kDa HA-MA (50% DoS) and 150 mg/ml of 50 kDa RCP-SH (50% DoS), the outcome was positive as the intact lenticules could be successfully removed from the molds (FIG. 6C). The lenticules obtained on continuously printing for 10 min required only a small change in temperature intermittently to obtain the same output. The printing parameters were adjusted accordingly to achieve the laminar flow during bioprinting. The importance of laminar flow in the bioprinting process is represented diagrammatically in FIG. 6D. The printed lenticule, as shown in FIGS. 6A-6C were approximately 400 microns thick and 14 mm in diameter.

TABLE 2
Lenticules printed with methyl cellulose
(14 kDa, ~30% DoS) as a thickener

| | | | Thickener | |
|---|---|---|---|---|
| | | | Concen- | |
| | | | tration | |
| S. No. | Polymers | Thickener | (mg/ml) | Outcome |
| | HA-MA (250 kDa, | Methyl | 0 | Lenticules of |
| 1 . | DoS 30%, 30 mg/ml) | Cellulose | 30 | irregular shape |
| | RCP (50 kDa, DoS | | 40 | |
| | 50%, 80 mg/ml) | | | |

TABLE 3
Lenticules printed with gelatin (medium bloom, 40-50 kDa) as a
thickener

| | | | T (° C.) at | |
|---|---|---|---|---|
| | | | which the | |
| S. | | | bio-ink is | |
| No. | Polymers | Thickener | printed | Outcome |
| 1. | HA-MA (50 kDa, DoS | Gelatin | 20 | Good printability |
| | 50%, 50 mg/ml) | (60 | 25 | but lenticules |
| | RCP (50 kDa, DoS | mg/ml) | 27 | were too brittle |
| | 50%, 80 mg/ml) | | 37 | The ink flowed to |
| | | | | the bottom of the |
| | | | | mold |
| | HA-MA (50 kDa, DoS | Gelatin | 22.5 | Good printability |
| 2. | 50%, 35 mg/ml) | (60 | 25 | Obtained intact |
| | RCP (50 kDa, DoS | mg/ml) | | lenticules |
| | 50%, 150 mg/ml) | | 37 | The ink flowed to |
| | | | | the bottom of the |
| | | | | mold |

As can be observed from Table 2 and 3, that gelatin was the preferred thickener to be used in the desired bio-ink formulations. In Table 3, observing the printing temperatures described therein, it can be appreciated that the temperatures in which the bio-ink was printed played a crucial role. The temperatures of 22.5° C., and 25° C. provided the desired bio-printed corneal lenticule. Whereas, at higher temperatures, the ink was not printable (FIG. 6A). Further, it can be observed that the bio-printed corneal lenticules obtained by using 35 mg/ml of HA-MA (50 kDa, DoS 50%) and 150 mg/ml of RCP (50 kDa, DoS 50%) provided the best results.

### Compressive Modulus of the Bio-Printed Corneal Lenticule

Similar to the modifications mentioned in the Example 1 in preparing the bio-ink formulations, the effect of different modes of the addition of photo-initiator (eosin) into the polymer solution, on the physical property (compression modulus) of the final product (bio-printed corneal lenticule) was assessed.

The compression study was conducted using the BiSS mechanical tester, at a rate of 1 mm/min up to a maximum strain of 50%. The compression modulus, in turn, were calculated from the slope of the linear region (0.1 to 0.2 mm/mm strain) on the stress (kPa) versus strain (millimeter per millimeter) curves.

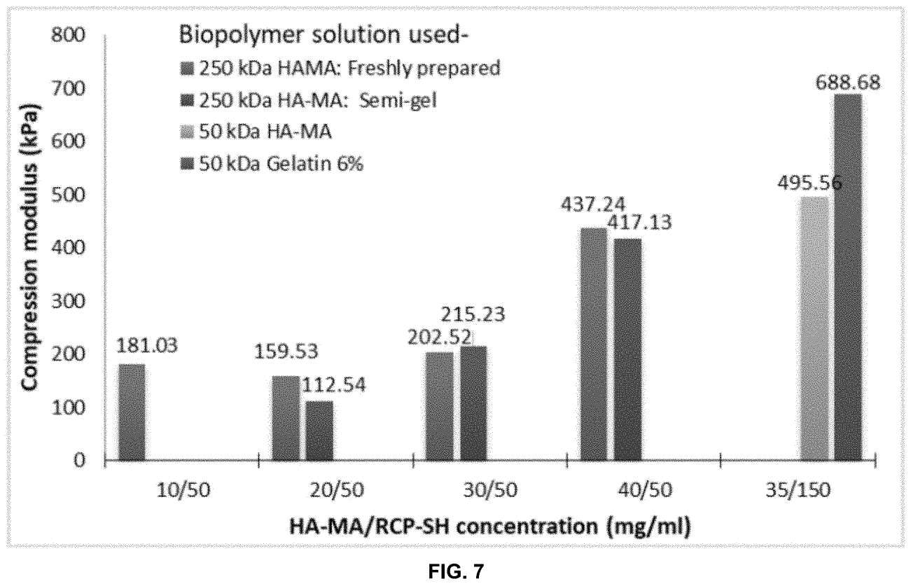

The addition of eosin overnight to the components did not produce a substantial increase in compression modulus when compared to the samples where eosin was added just prior to hydrogel formation (FIG. 7). The highest compression modulus was observed for 40 mg/mL of 250 kDa HA-MA while 20 and 30 mg/mL of HA-MA was found to be more elastic but showed lower compression modulus, than that of the native cornea (˜300 kPa).

The compression modulus of the hydrogel having 50 kDa HA-MA and RCP-SH in 35/150 mg/ml concentration increased on the addition of gelatin (medium bloom, 40-50 kDa, 60 mg/ml). This study also depicts that after removal of the thickener from the printed lenticule, the modulus would still be sufficient enough to maintain its integrity and perform the required physical functions. Based on the above-discussed screening experiments, the use of gelatin as a thickener in the HA-MA (50 kDa) and RCP-SH based bio-ink provided reproducibility in printability along with fulfilling the material requirements. Thus, further characterization was performed using 50 kDa HA-MA/RCP-SH (35/150 mg/mL) and 60 mg/ml gelatin hydrogels/bio-printed lenticules as one of the possible embodiments, although other combinations have also been described.

### Example 4

### Physico-Chemical Characterization of Bio-Ink Comprising of 50 kDa HA-MA (50% DoS, 35 mg/ml) with 50 kDa RCP-SH (50% DoS, 150 mg/mL) and Gelatin (Medium Bloom, 40-50 kDa, 60 mg/ml)

The present Example describes the different parameters for the bio-ink formulation comprising 35 mg/ml of 50 kDa HA-MA (50% DoS), 150 mg/ml of 50 kDa RCP-SH (50% DoS), and 60 mg/ml of gelatin (medium bloom—40-50 kDa), wherein the bio-ink formulation was prepared using the method as described in FIG. 1C and Example 2.

Although the present Example describes the bio-ink formulations and respective bio-printed corneal lenticule obtained by the bio-ink formulations comprising 35 mg/ml of 50 kDa HA-MA (50% DoS), 150 mg/ml of 50 kDa RCP-SH (50% DoS), and 60 mg/ml of gelatin (medium bloom—40-50 kDa), it can be contemplated that bio-ink formulations comprising a modified hyaluronic acid having a molecular weight in the range of 30-300 kDa, and with a degree of substitution in the range of 10-80%; a modified collagen peptide having a molecular weight in the range of 10-80 kDa, and with a degree of substitution in the range of 10-80%; and gelatin having a bloom value in the range of 50-325, wherein gelatin is in the concentration range of 0.1-150 mg/ml with respect to the bio-ink formulation can be used. A person skilled in the art can use any formulation falling within the above-mentioned ranges. The formulations not providing good results have already been explained in Tables 2 and 3. For example, the bio-ink formulations comprising:

40 mg/ml of 50 kDa HA-MA (50% DoS); 100 mg/ml of 50 kDa RCP (50% DoS); and 20-100 mg/ml of medium bloom gelatin; or

45 mg/ml of 50 kDa HA-MA (50% DoS); 120 mg/ml of 50 kDa RCP (50% DoS); and 30-75 mg/ml of medium bloom gelatin; or

35 mg/ml of 50 kDa HA-MA (50% DoS); 90 mg/ml of 50 kDa RCP (50% DoS); and 60 mg/ml of medium bloom gelatin; or

32 mg/ml of 50 kDa HA-MA (50% DoS); 180 mg/ml of 50 kDa RCP (50% DoS); and 30-75 mg/ml of medium bloom gelatin; or can be used to obtain the desirable bio-ink formulation and the respective bio-printed corneal lenticule. Similarly, the molecular weight of the polymers can also be varied to suit the needs.

### Transmittance Studies

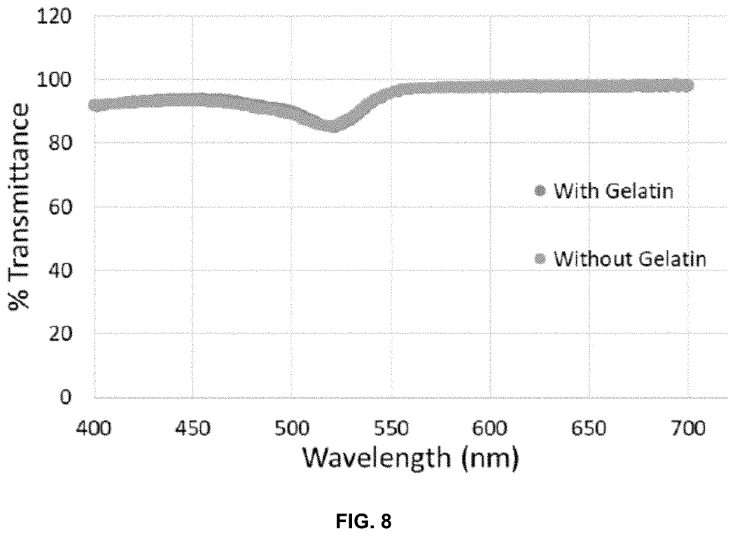

For transmittance studies, the bio-ink formulation obtained using the method described in FIG. 1C and explained in Example 2. The bio-ink formulation was prepared using 35 mg/ml of 50 kDa HA-MA (50% DoS), 150 mg/ml of 50 kDa RCP-SH (50% DoS), and 60 mg/ml of gelatin (medium bloom—40-50 kDa). As a control, the bio-ink formulation without using gelatin was also prepared. The bio-ink was poured inside the wells of a 96 well plate (n=3) and exposed to light to form the hydrogel. The hydrogel as used herein refers to the cross-linked form of the bio-ink, wherein the cross-linking was done by exposing to white light having an intensity in the range of 100-150 mW/cm². Absorbance scanning was done in the range of 350-750 nm using saline of equal volume as hydrogels as blank. Finally, the absorbance reading was converted to transmittance using the formula—% T=10\hat{ }(2−Abs) and represented in graph (as per the protocol mentioned in Wang et al., 2015. Biomacromolecules 2014, 15, 9, 3421-3428. https://doi.org/10.1021/bm500969d). The hydrogels irrespective of the presence or absence of gelatin showed 85-99% transmittance to the visible light relative to 1×PBS (FIG. 8). The average transmittance value remained >94% for both the study groups in the visible light range (Table 4).

TABLE 4
Average transmittance values

| | 50 kDa HA- | 50 kDa RCP- | | % |
|---|---|---|---|---|
| S. No. | MA (mg/ml) | SH (mg/ml) | gelatin | transmittance |
| 1 | 35 | 150 | 6 | 94.67% |
| 2 | 35 | 150 | 0 | 94.63% |

### Swelling Profile Studies

The swelling study was performed on the bio-printed lenticule as per the method published in Sani, E. S. et al., 2019. Sutureless repair of corneal injuries using naturally derived bio-adhesive hydrogels. Science advances, 5(3), p.eaav1281.

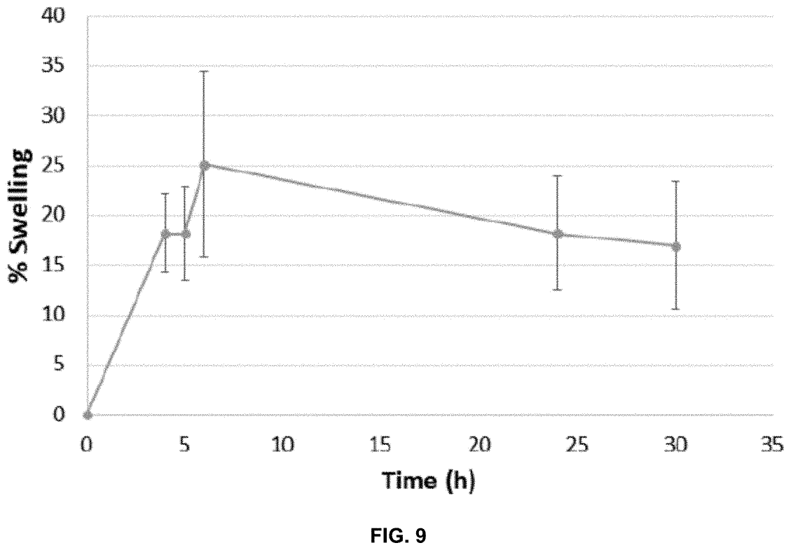

The result is shown in (FIG. 9). The maximum swelling of the hydrogel on being incubated in PBS was observed to be 25.07% within 6 h, after which it did not swell further. Subsequently, there was an evident loss in weight in all the replicates, which can be attributed to the release of gelatin from the hydrogel.

### Gelatin Release Profile

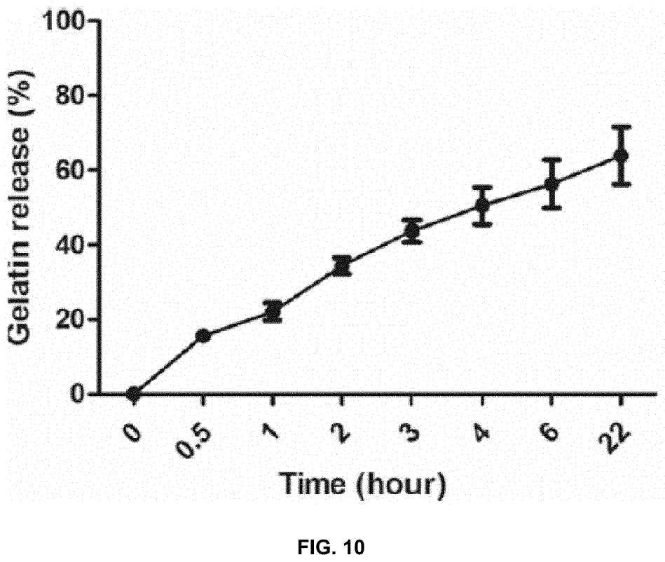

The gelatin release profile was studied (based on the method published in Raut et al., 2019. Journal of Materials Science volume 54, pages 10457-10472 https://doi.org/10.1007/s10853-019-03643-0) on the bio-printed corneal lenticule obtained from the bio-ink formulation been studied in the present Example. The release of gelatin from bio-printed lenticules followed a phase-release profile, wherein in the 1^{st} phase it had a burst release pattern in the first 30 min after which there was a gradual increase for 3 h followed by a steady reduction and eventually stable from 6 h onwards. In total, 63.9% gelatin was released from the lenticule in 22 h (FIG. 10). The obtained result is normalized values from the HA-MA/RCP-SH (35/150 mg/mL) hydrogel without gelatin to avoid any interference in quantification from the protein component (RCP-SH) release.

### Biodegradation Study

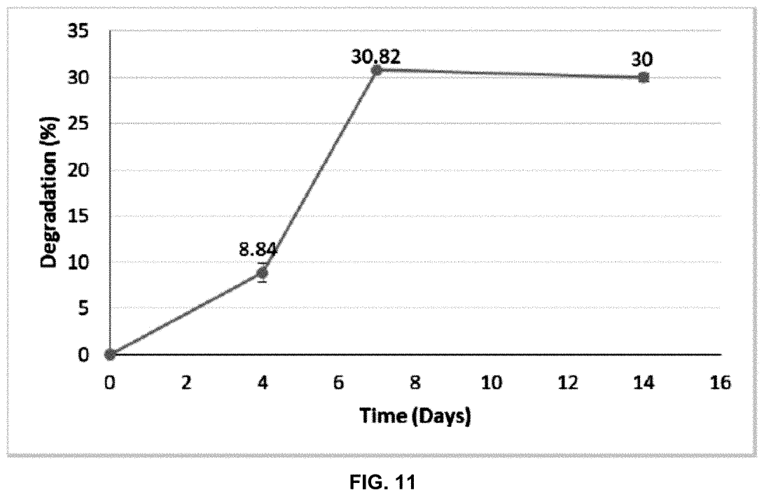

FIG. 11 depicts the degradation profile of the hydrogel of the bio-ink formulation. Briefly, the hydrogels of definite volume were prepared, lyophilized, and weighed (Wi). Replicate hydrogels were then incubated in PBS or saline of pH ˜7.4 at 37° C. and shaken in an orbital shaker. At specific time points, hydrogels were taken out, lyophilized, and weighed (Wd) and the mass was calculated as Weight loss or degradation (%)=(Wi−Wd)/Wi×100 (Li 2006, Biomaterials https://dx.doi.org/10.1016%2Fj.biomaterials.2005.07.019)

The degradation of hydrogel was slow initially for 3 days after which the rate increased, and 30.8% of the hydrogel mass was degraded by day 7, and the rate remained almost stable after that (FIG. 11).

### Biocompatibility—In-Vitro Studies

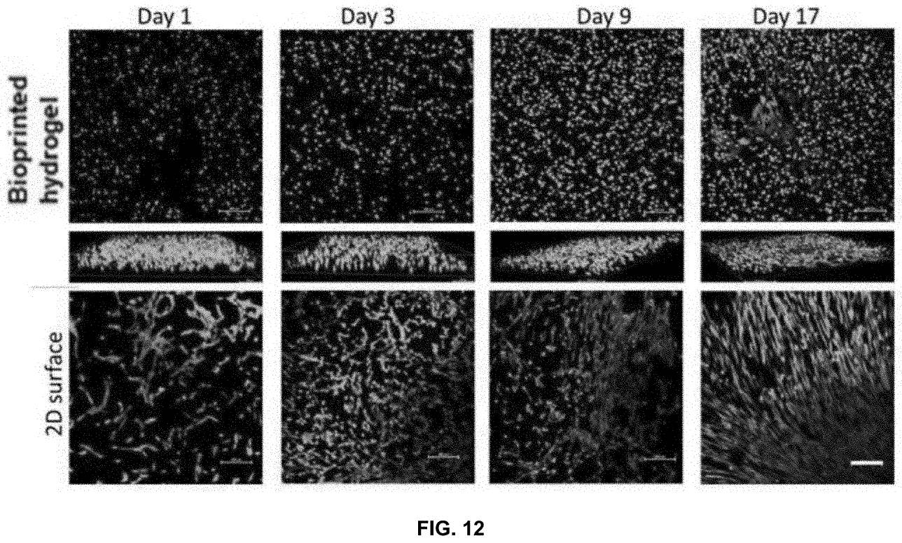

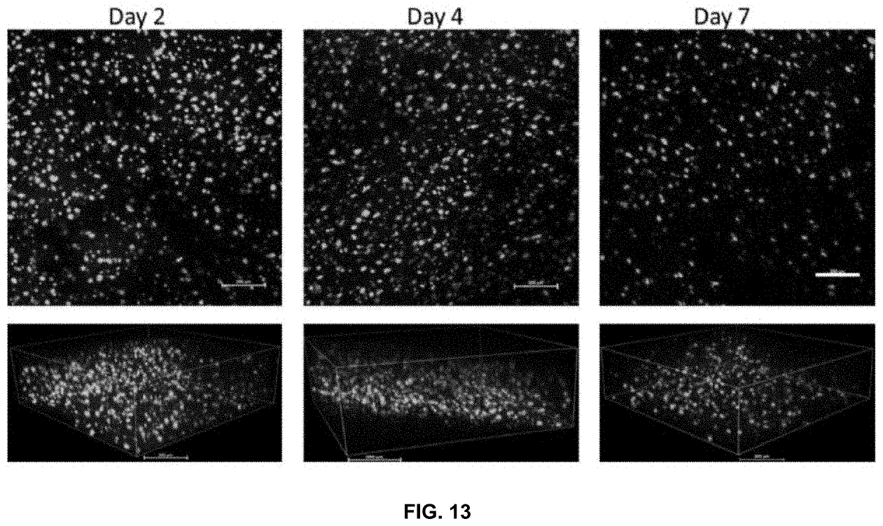

The prepared hydrogel formulations were assessed for their suitability to elicit corneal tissue regeneration by culturing donor-derived CLSCs (3 million cells per ml) (FIG. 12) and BM-MSCs (FIG. 13) encapsulated inside the pre-gel mix and bioprinting the ink with the cells. Cells were homogeneously distributed within the hydrogel whereby ˜80% encapsulated cells were alive throughout the culture duration. The population of cells showing elongated morphology started appearing from day 9 and increased slightly thereafter in the bio-ink comprising CLSCs. Some encapsulated cells migrated towards the bottom and have formed a monolayer on the hydrogel surface. Whereas, in the BM-MSCs culture, the elongated morphology for some cells was observed on day 7.

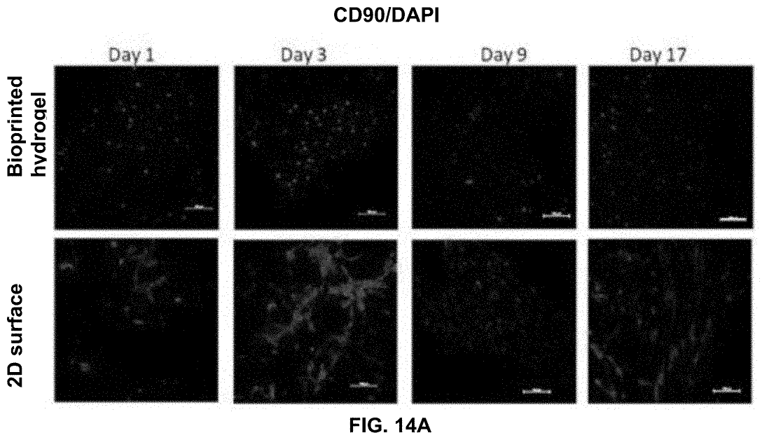

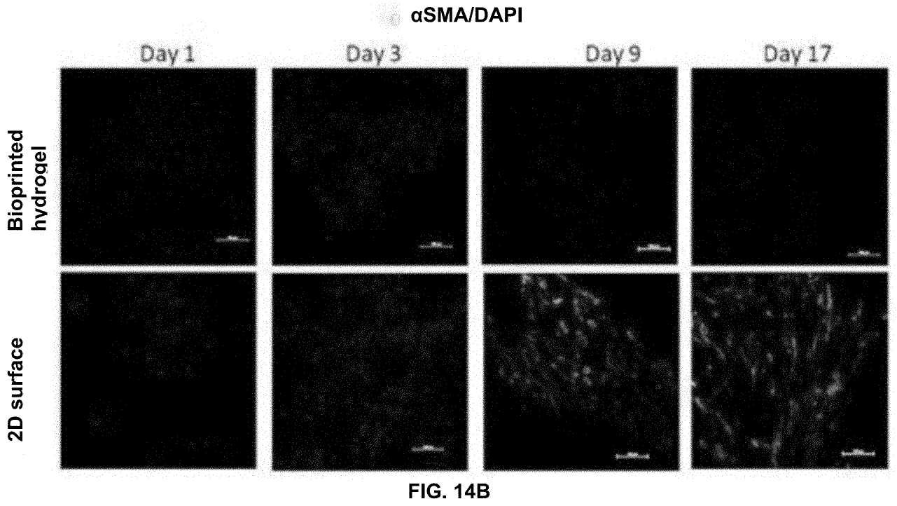

For complete tissue regeneration at the defect site, it is of utmost importance that the stromal stem cells maintain their phenotype and help in scar-less healing of the wound while gradually attaining the differentiated state. In the in-vitro condition, this process of gradual differentiation can be assessed by checking the expression of biomarkers that are specific to a particular stage of cells' life cycle. CD90 is one such biomarker that is expressed by the stromal stem cells, whereas the expression of αSMA by the cells would reflect their differentiated state to keratocytes or myofibroblasts. The obtained results (FIGS. 14A and 14B) shows that bio-ink comprising CLSCs, when maintained in culture for 17 days, expressed CD90 and did not express αSMA. Whereas, the cells cultured on the 2D surface showed prominent expression of αSMA, indicating their differentiated phenotype. The results, as depicted herein, provides a significant advantage to the bio-printed lenticules (obtained from the bio-ink formulation of the present Example) in terms that they can suppress myofibroblast differentiation and hence have the potential to support scar-less wound healing of the corneal tissue.

### Example 5

### Physico-Chemical Characterization of Bio-Ink Comprising 50 kDa HA-MA (50% DoS) with Collagen MA (250 kDa ColMA, DoS 29%)

Although the present Example describes the bio-ink formulations and respective bio-printed corneal lenticule obtained by the bio-ink formulations comprising 50 kDa HA-MA (50% DoS), 250 kDa Col-MA (29% DoS) (without gelatin), it can be contemplated that bio-ink formulations comprising a modified hyaluronic acid having a molecular weight in the range of 30-300 kDa, and with a degree of substitution in the range of 10-80%; a modified collagen having a molecular weight in the range of 200-300 kDa, and with a degree of substitution in the range of 10-80%; and gelatin having a bloom value in the range of 50-325, wherein gelatin is in the concentration range of 0.1-150 mg/ml with respect to the bio-ink formulation can be used. The hydrogel formulations without gelatin is giving desirable results in terms of transmittance and biocompatibility (results of the present Example), a person skilled in the art can use any formulation falling within the above-mentioned ranges. However, gelatin is required for attaining the desirable viscosity of the bio-ink formulations to be able to print the same to obtain the bio-printed corneal lenticule. Therefore, the bio-ink formulations comprising:

60 mg/ml of 50 kDa HA-MA (50% DoS); 10 mg/ml of 250 kDa Col-MA (29% DoS); and 20-100 mg/ml of medium bloom gelatin; or

80 mg/ml of 50 kDa HA-MA (50% DoS); 20 mg/ml of 250 kDa Col-MA (29% DoS); and 40-100 mg/ml of medium bloom gelatin; or

60 mg/ml of 50 kDa HA-MA (50% DoS); 10 mg/ml of 250 kDa Col-MA (29% DoS); and 20-100 mg/ml of medium bloom gelatin; or

10 mg/ml of 50 kDa HA-MA (50% DoS); 5 mg/ml of 50 kDa Col-MA (50% DoS); and 30-75 mg/ml of medium bloom gelatin; or can be used to obtain the desirable bio-ink formulation and the respective bio-printed corneal lenticule.

### Transmittance

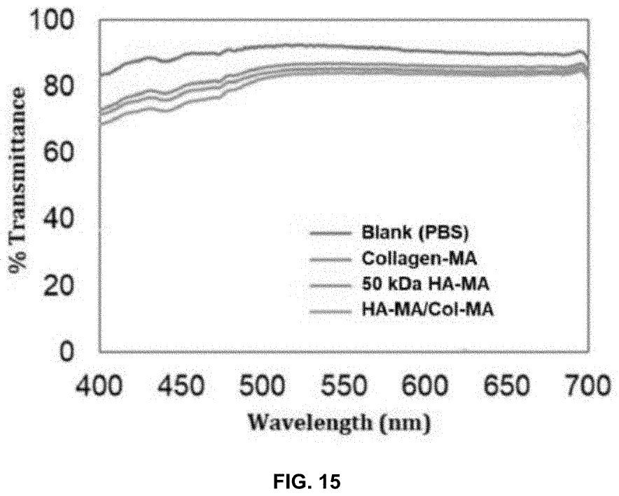

An important property for any cornea mimetic material for fulfilling functional requirements is light transmittance. FIG. 15 shows the transmittance of the light in visible range (400-700 nm) by a representative bio-ink with respect to the individual components, where 1×phosphate buffer saline (PBS) was used as blank. It is evident from the results that the individual components and the combination, i.e., HA-MA/ColMA bio-ink in 50/9 mg/ml concentration, show comparable transmittance with PBS.

### Bio-Compatibility—In-Vitro Studies

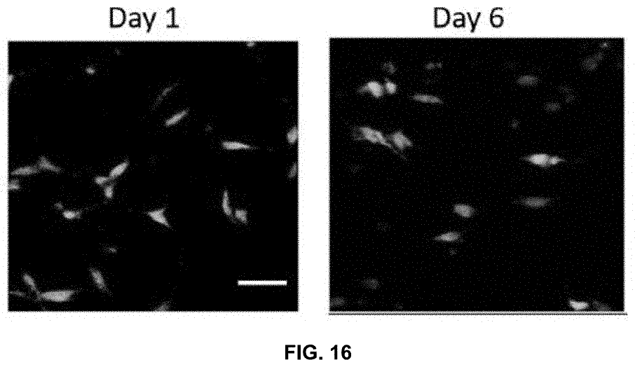

The CLSCs were encapsulated inside a hydrogel (50 mg/ml of 50 kDa HA-MA with 50% DoS, and 9 mg/ml of 250 kDa Col-MA with 29% DoS) to check the compatibility of the polymer mix and crosslinking process with the cells found in native human cornea. FIG. 16 represents the staining for live and dead cells. The result showed that the hydrogel matrix is highly cytocompatible, and the composition supports the attachments of the encapsulated cells.

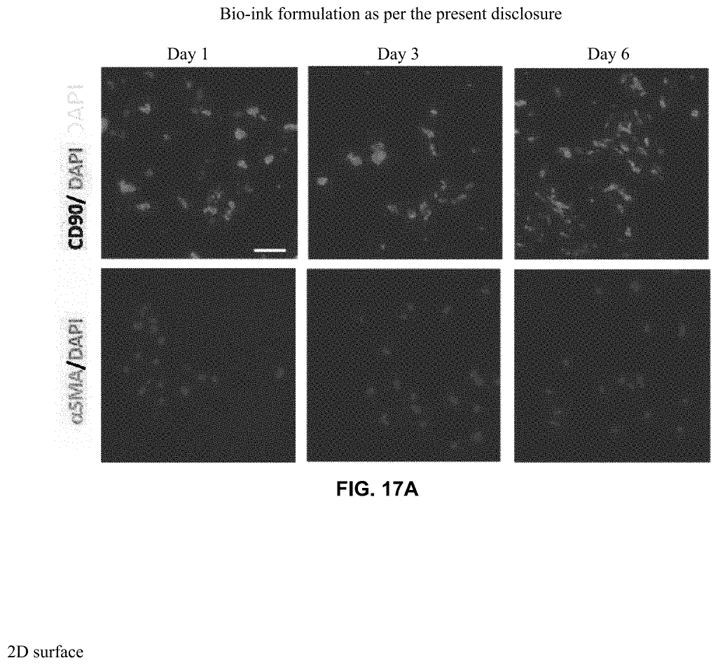

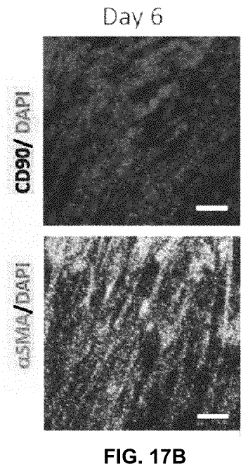

For complete tissue regeneration at the defect site, it is of utmost importance that the stromal stem cells maintain their phenotype and help in scar-less healing of the wound while gradually attaining the differentiated state. In in vitro condition, this process of gradual differentiation can be assessed by checking the expression of biomarkers that are specific to a particular stage of cells' life cycle. CD90 is one such biomarker that is expressed by the stromal stem cells, whereas the expression of αSMA by the cells would reflect their differentiated state to keratocytes or myofibroblasts. The obtained results (FIGS. 17A-17B) show that CLSCs cultured in the HA-MA/ColMA bio-ink (50 mg/ml of 50 kDa HA-MA with 50% DoS, and 9 mg/ml of 250 kDa Col-MA with 29% DoS) were showing better expression of CD90 and weak expression of αSMA. Whereas, the cells cultured on the 2D surface (refers to a glass coverslip) showed prominent expression of αSMA within 6 days in culture, indicating their differentiated phenotype. The HA-MA/ColMA bio-ink suppressed myofibroblast differentiation and hence have the potential to support scar-less wound healing of the corneal tissue.

### Example 6

### Studies with Bio-Ink Formulation Comprising “33 kDa” HA-MA (50% DoS)/50 kDa RCP-SH (50% DoS)

The prepared hydrogel formulations having 75/125 and 75/150 mg/ml concentration ratios of 33 kDa HA-MA (50% DoS) and 50 kDa RCP-SH (50% DoS), were assessed for their suitability to elicit corneal tissue regeneration. The hydrogels as per the present Example were prepared without gelatin, however, it can be contemplated that the hydrogels with gelatin would also provide similar results. Firstly, the re-epithelialization capability using the limbal or corneal epithelial cells (LECs or CECs) on hydrogel surfaces were studied. Secondly, to demonstrate stromal regeneration, CLSCs were encapsulated inside the hydrogels, and their viability, proliferation capacity, and phenotype were studied in vitro.

### Re-Epithelialization Studies

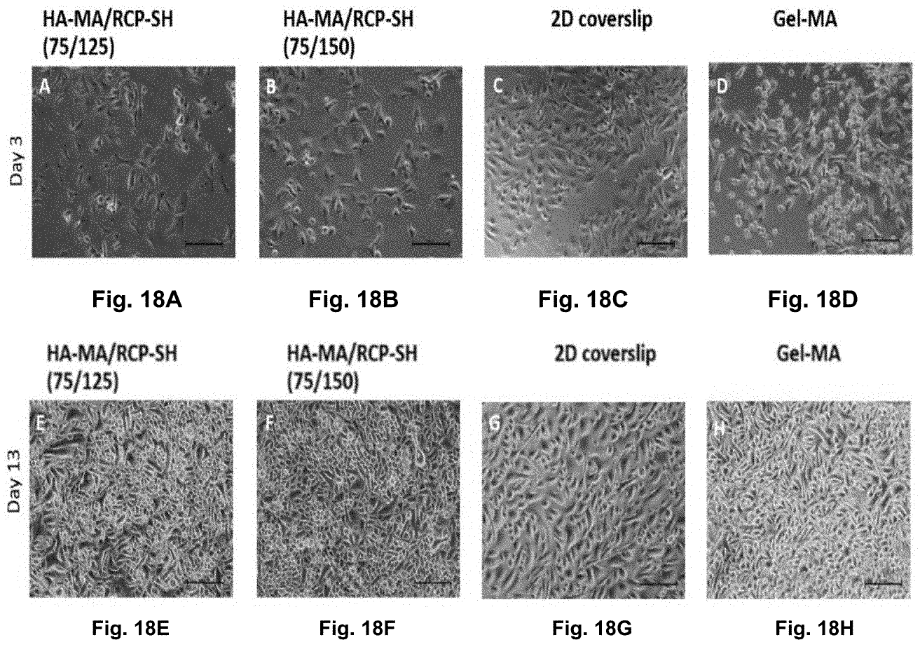

To demonstrate biocompatibility of the HA-MA/RCP-SH hydrogel formulation (“33 kDa”, formulations containing HA-MA (33 kDa)/RCP-SH (50 kDa) in the ratios 75/125 and 75/150 mg/ml), primary human CECs were seeded and cultured on the surface of the hydrogel. The epithelial cells adhered and proliferated on the surface of the hydrogels yielding a confluent monolayer by the end of two weeks (FIGS. 18A, 18B, 18E, 18F). This observation was in comparable to the 2D coverslip surface (FIGS. 18C, 18G) and Gel-MA (200 mg/ml; FIGS. 18D, 18H) hydrogel, which were used as positive control. This data demonstrates that HA-MA/RCP-SH hydrogels act as a cornea-mimetic bio-engineered material that can promote corneal wound healing/regeneration in vivo.

### Stromal Regeneration: Encapsulation of CLSCs in the Hydrogel

The compatibility of the hydrogels to the CLSCs, which would ultimately indicate the stromal regeneration capability of the hydrogel, was assessed by culturing CLSCs on the hydrogel surface, followed by encapsulation studies.

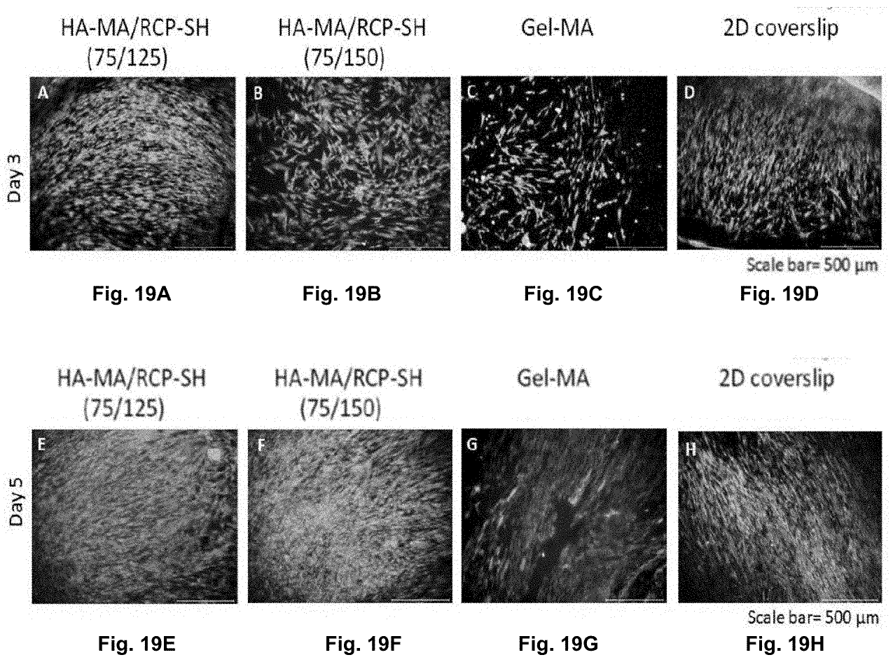

FIGS. 19A, 19B, 19E, 19F represent the viability assessment of the CLSCs when cultured on the hydrogel surface for 5 days. As evident from the results, the cells showed rapid proliferation and covered the hydrogel surface within 5 days. Also, the viable cell population, marked by cell cytoplasm stained in green color, shows that the culture environment is compatible for the cells to proliferate. The cell growth on the HA-MA/RCP-SH hydrogel surface was higher than on Gel-MA (FIGS. 19C, 19G), whereas, it was similar to the cells on coverslips (FIGS. 19D, 19H), which was used as a positive control.

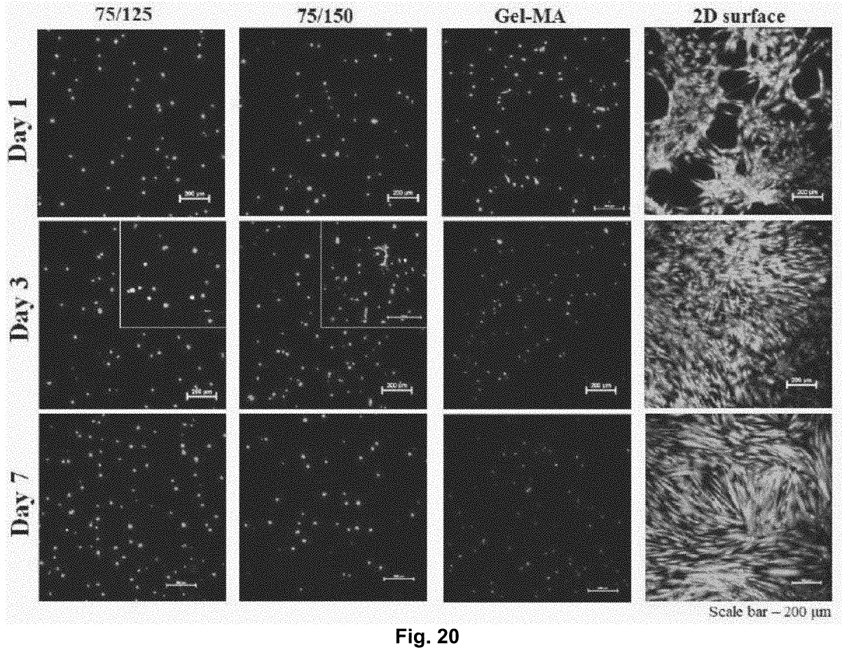

Viability of CLSCs was also assessed for a week on encapsulating the cells in the hydrogel matrix. The cells appearing green (due to calcein-AM uptake by live cells) in FIG. 20, represents the live cell population. The CLSCs encapsulated in the HA-MA/RCP-SH hydrogels were viable throughout the culture duration, and the viable population was similar to the 2D cover slip and was higher than the Gel-MA (20% w/v or 200 mg/ml with DoS of more than 95%). Also, the insets in day 3 images for the HA-MA/RCP-SH hydrogels show that some cells have started to attain the elongated morphology, which was shown by cells cultured on the 2D surface. FIGS. 21A-21B show that CLSCs cultured in the HA-MA/RCP-SH hydrogel matrix were showing better expression of CD90 (FIG. 21A) and weak expression of αSMA (FIG. 21B) while cells cultured on the 2D surface showed prominent expression of αSMA (FIG. 21B), indicating their differentiated phenotype. HA-MA/RCP-SH hydrogels suppressed myofibroblast differentiation and hence have the potential to support scar-less wound healing of the corneal tissue.

### Example 7

### Bio-Ink and the Respective Bio-Printed Corneal Lenticule Comprising Exosomes

As an implementation of the present disclosure, the bio-ink formulations and the respective bio-printed corneal lenticule comprising exosomes in the presence of stem cells along with the polymers HA-MA, and RCP-SH, and thickener gelatin is provided herewith. It can be appreciated by previous examples that the bio-ink formulations, and the hydrogels allow the growth of the stem cells and are bio-compatible, therefore the inclusion of exosomes is contemplated to assist the growth of the stem cells and help in the healing of the wound in a scar-less manner.

As another implementation, the bio-ink formulations, and respective bio-printed corneal lenticule comprising polymers HA-MA, and RCP-SH, and thickener gelatin along with exosomes in the absence of stem cells are also disclosed herewith and is contemplated to provide desirable results.

### Example 8

### Comparison of the Bio-Ink Formulation of the Present Disclosure with the Known Bio-Ink Formulation

The present Example compares certain parameters of the bio-ink formulation as disclosed in the present disclosure with that of the known bio-ink formulations.

TABLE 5
Comparative analysis

| | | | | | Bio-printed corneal | |
|---|---|---|---|---|---|---|
| | | | | | lenticule of the present | |
| | | | | | disclosure prepared from | |
| | | | | | bio-ink formulation | |
| | | | | | HA-MA (50 kDa, 50% | |
| | Hydrogel obtained from Polyvinyl | | | | DoS, 35 mg/ml) and RCP- | |
| | alcohol (13% w/v, 89-98 kDa) + chitosan | | | | SH (~51 kDa, 50% DoS, | |
| | (0-5% w/v, 50-190 kDa) as published in | | | | 150 mg/ml) | |
| | Ulag et al. 3D printed artificial cornea for | | | | With 60 | |
| | corneal stromal transplantation. Euro Pol | | | | mg/ml | |
| | J. 2020;133;109744. | | | | Gelatin | |
| | | 13%PVA/ | 13%PVA/ | 13%PVA/ | (175 | |
| Parameters | 13% PVA | 1% CS | 3% CS | 5% CS | Bloom) | w/o Gelatin |
| Maximum | 305% | 530% | 540% | 560% | 25.07% | N/A |
| Swelling | | | | | | |
| Biodegradation | 90% | 120% | 127% | 140% | 30.82% | N/A |
| (PBS)- | | | | | | |
| Day 7 | | | | | | |
| Transmittance | 61% | 56% | 53% | 49% | 94.67% | 94.63% |
| Tensile | 40.28 | 11.65 | 12.72 | 8.94 | 8.46 | N/A |
| strength (kPa) | | | | | | |

| | | | |
|---|---|---|---|
| Compressive | N/A | 627.97 | 495.56 |
| modulus (kPa) | | | |

It can be observed from Table 5, that the bio-ink formulation disclosed in the present disclosure leads to a far more superior hydrogel/bio-printed corneal lenticule having a controlled swelling, lesser degradation, and higher transmittance as compared to the artificial cornea published in the mentioned prior art (Ulag et. al., 2020).

### Example 9

### Methods for Culturing Stem Cells, Obtaining Purified Exosomes

The present disclosure also discloses the aspect of culturing stem cells in a two-dimensional or three-dimensional manner so as to obtain large quantity of expanded stem cells, and conditioned medium for biomedical applications.

The conditioned medium was used to purify high-quality exosomes. The exosomes thus obtained were used in the bio-ink formulations as disclosed in the present disclosure.

The cell culturing method also included priming the mesenchymal stem cells with the conditioned medium derived from culturing of corneal limbal stem cells (referred to as corneal stromal stem cell-derived conditioned medium), the conditioned medium of mesenchymal stem cells obtained by the priming method were used for purifying the exosomes to be used in the bio-ink formulations of the present disclosure.

The aspects of the culturing of stem cells for obtaining expanded stem cells, and obtaining cell culture-conditioned medium, further, the process for obtaining exosomes from the secretome of the conditioned medium are disclosed in the PCT Applications; PCT/IN2020/050622, and PCT/IN2020/050623 which are incorporated in its entirety in the present disclosure.

### Example 10

### Methods of treating a Subject with Corneal Defects

The bio-printed corneal lenticule as disclosed in the present disclosure, can be further used for treating subjects with corneal defects. The corneal defects or disorders can be selected from the group consisting of infectious keratitis, inflammatory disorders, inherited corneal epithelial-stromal dystrophies, degenerative conditions and trauma-induced injuries. The corneal disorders which can cause corneal blindness can be treated with the bio-printed corneal lenticule as disclosed in the present disclosure. The method comprises implanting the bio-printed corneal lenticule to the subject in need thereof. The bio-printed corneal lenticule can be sutured in patients as per the methods mentioned in Islam, et al. Biomaterials-enabled cornea regeneration in patients at high risk for rejection of donor tissue transplantation. npj Regen Med 3, 2 (2018). https://doi. org/10.1038/ s41536-017-0038-8.

The method of treatment may or may not include a step of providing a formulation comprising: comprising: (i) exosomes selected from the group consisting of corneal stromal stem cell derived-exosomes, primed mesenchymal stem cell derived-exosomes, and naive mesenchymal stem cell derived-exosomes; and (ii) a clinically approved eye drop formulation. The clinically approved eye drop formulation may be selected from the group consisting of the following:

1. Tearhyl® (Sodium hyaluronate, 0.1-0.3% solution)
2. Refresh Optive® (Carboxymethylcellulose, 0.5% solution)
3. Systane Ultra® (Polyethylene glycol, MW 400, 0.4% solution)
4. Leader® Artificial Tears Solution (Polyvinyl alcohol, 1.4% solution)
5. Systane Balance® (Propylene glycol, 0.6% solution)
6. MIKELAN® LA (Alginate based)

Further, the method of treatment may involve providing the formulation as a stand-alone treatment option.

### Advantages of the Present Disclosure

The present disclosure provides a bioengineered bio-ink and a bio-printed corneal stromal lenticule which have the desirable features of being bio-mimetic, bio-compatible, and bio-degradable. Also, the bio-ink formulation, as described herein, is in the preferred viscosity range to allow the ease of 3D printing to obtain the bio-printed corneal lenticule. The bio-ink as well as the bio-printed corneal lenticule promote scar-less corneal healing, thereby, resulting in transparent cornea post-transplant as described in the present disclosure. One other significant advantage is that the present disclosure discloses a transparent suturable 3D bio-printed corneal lenticule using hyaluronic acid, which is a natural component present in the eye can serve as a viable therapeutic option for replacement of diseased/injured corneas with partial or full-thickness transplant grafts. Thus, the bio-printed corneal lenticule is a bio-mimetic lenticule, which is certainly advantageous in terms of the treatment. The inclusion of exosomes and/or stem cells in the bio-printed corneal lenticule can further assist in providing regenerative treatment options for subjects with extensive corneal defects. The bio-printed corneal lenticule as described herein, along with the process of three-dimensional cell culture as disclosed in PCT Application No. PCT/IN2020/050622 along with the priming aspect of the mesenchymal stem cells as disclosed in the PCT Application No. PCT/IN2020/050623 can potentially cater to the requirements of many subjects in need thereof.

Since the bio-printed corneal lenticule is bio-mimetic, it can also be used as a model for studying drug toxicity. Also, the lenticule can also be used to study and better understand various corneal diseases/defects and help in the advancement of research. The bio-printed corneal lenticule as disclosed herein can be used as a tool to study toxicity of the drugs and also as a tool for understanding disease progression and mechanism (disease modelling).

The present disclosure discloses bioengineered bio-ink formulations and a bio-printed corneal lenticule which have the desirable features of being bio-mimetic, bio-compatible, and bio-degradable. The bio-ink formulation comprising: (a) a first polymer selected from the group consisting of modified hyaluronic acid, modified polyethylene glycol, modified polyvinyl alcohol, modified poly(N-isopropylacrylamide), modified alginate, silk, and modified silk; (b) a second polymer selected from the group consisting of collagen peptide, modified collagen peptide, collagen, and modified collagen; and (c) a thickener selected from the group consisting of gelatin, modified cellulose, gellan gum, xanthum gum, polyethylene glycol, poloxamer, polyvinyl alcohol, and alginate, having a viscosity in the range of 1690-5300 cP is disclosed in the present disclosure.

The bio-ink formulation comprising: a modified hyaluronic acid having a molecular weight in the range of 30-100 kDa, and with a degree of substitution in the range of 30-70%; a modified collagen peptide having a molecular weight in the range of 30-70 kDa, and with a degree of substitution in the range of 30-70%; and gelatin having a bloom value in the range of 50-325, and having a concentration range of 0.1-150 mg/ml with respect to the bio-ink formulation is also disclosed herein. The bio-ink formulations further comprise a photo-initiator (0.5-1×eosin) for initiating cross-linking of the polymers. A white light of certain intensity is illuminated on the bio-ink to further complete the process of cross-linking. The bio-ink formulations further comprise stem cells selected from the group consisting of human bone marrow-mesenchymal stem cell, adipose tissue-mesenchymal stem cell, umbilical cord-mesenchymal stem cell, Wharton jelly-mesenchymal stem cell, dental pulp-derived mesenchymal stem cell, and corneal limbal stem cell-derived conditioned media primed mesenchymal stem cells. Further, the bio-ink formulations also comprise exosomes selected from the group consisting of corneal stromal stem cell derived-exosomes, primed mesenchymal stem cell derived-exosomes, and naive mesenchymal stem cell derived-exosomes. The bio-ink formulations can also comprise exosomes without the presence of the stem cells. Also, disclosed herein is a process for obtaining the bio-ink formulation. Further, the present disclosure provides a bio-printed corneal lenticule comprising the bio-ink formulation as described herein. The bio-printed corneal lenticule obtained from the above-mentioned bio-ink formulation provides a controlled swelling, and a high tensile strength. Further, the bio-printed corneal lenticule is also resistant to bio-degradation (2-40% within 30 days under in-vitro conditions) and exhibits transmittance of more than 93%. The bio-printed corneal lenticule thus obtained promotes the growth of stem cells (bio-compatible), and also promotes epithelialization and stromal regeneration, thus providing an opportunity to heal the scars in the corneal tissue. Also, the bio-printed corneal lenticule and/or the hydrogels suppressed myofibroblast differentiation and hence have the potential to support scar-less wound healing of the corneal tissue. The presence of exosomes in the hydrogels/corneal lenticule further supports the regenerative treatment. Thus, the present disclosure provides the bio-ink formulations and corresponding bio-printed corneal lenticule with desired characteristics of tensile strength, compressive modulus, transmittance, controlled swelling and resistant to degradation which can be used to treat corneal defects in a subject to promote scar-less wound healing.

The bio-ink formulation comprising: a modified hyaluronic acid having a molecular weight in the range of 40-70 kDa, and with a degree of substitution in the range of 30-70%; a modified collagen peptide having a molecular weight in the range of 30-70 kDa, and with a degree of substitution in the range of 20-70%; and gelatin having a bloom value in the range of 175-225, and having a concentration range of 40-80 mg/ml with respect to the bio-ink formulation is also disclosed herein. The bio-ink formulation further comprises a photo-initiator (0.5-1×eosin) for initiating cross-linking of the polymers. A white light of certain intensity is illuminated on the bio-ink to further complete the process of cross-linking. The bio-ink formulations further comprise stem cells selected from the group consisting of human bone marrow-mesenchymal stem cell, adipose tissue-mesenchymal stem cell, umbilical cord-mesenchymal stem cell, Wharton jelly-mesenchymal stem cell, dental pulp-derived mesenchymal stem cell, and corneal limbal stem cell-derived conditioned media primed mesenchymal stem cells. Further, the bio-ink formulations also comprise exosomes selected from the group consisting of corneal stromal stem cell derived-exosomes, primed mesenchymal stem cell derived-exosomes, and naive mesenchymal stem cell derived-exosomes. The bio-ink formulations can also comprise exosomes without the presence of the stem cells. Also, disclosed herein is a process for obtaining the bio-ink formulation. Further, the present disclosure provides a bio-printed corneal lenticule comprising the bio-ink formulation as described herein. The bio-printed corneal lenticule obtained from the above-mentioned bio-ink formulation provides a controlled swelling, and a high tensile strength. Further, the bio-printed corneal lenticule is also resistant to bio-degradation (2-40% within 30 days under in-vitro conditions) and exhibits transmittance of more than 93%. The bio-printed corneal lenticule thus obtained promotes the growth of stem cells (bio-compatible), and also promotes epithelialization and stromal regeneration, thus providing an opportunity to heal the scars in the corneal tissue. Also, the bio-printed corneal lenticule and/or the hydrogels suppressed myofibroblast differentiation and hence have the potential to support scar-less wound healing of the corneal tissue. The presence of exosomes in the hydrogels/corneal lenticule further supports the regenerative treatment. Thus, the present disclosure provides the bio-ink formulations and corresponding bio-printed corneal lenticule with desired characteristics of tensile strength, compressive modulus, transmittance, controlled swelling and resistant to degradation which can be used to treat corneal defects in a subject to promote scar-less wound healing.

The bio-ink formulation comprising: a modified hyaluronic acid having a molecular weight in the range of 30-50 kDa, and with a degree of substitution in the range of 30-70%; a modified collagen peptide having a molecular weight in the range of 30-70 kDa, and with a degree of substitution in the range of 20-70%; and gelatin having a bloom value in the range of 175-225, and having a concentration range of 40-80 mg/ml with respect to the bio-ink formulation is also disclosed herein. The bio-ink formulation further comprises a photo-initiator (0.5-1×eosin) for initiating cross-linking of the polymers. A white light of certain intensity is illuminated on the bio-ink to further complete the process of cross-linking. The bio-ink formulations further comprise stem cells selected from the group consisting of human bone marrow-mesenchymal stem cell, adipose tissue-mesenchymal stem cell, umbilical cord-mesenchymal stem cell, Wharton jelly-mesenchymal stem cell, dental pulp-derived mesenchymal stem cell, and corneal limbal stem cell-derived conditioned media primed mesenchymal stem cells. Further, the bio-ink formulations also comprise exosomes selected from the group consisting of corneal stromal stem cell derived-exosomes, primed mesenchymal stem cell derived-exosomes, and naive mesenchymal stem cell derived-exosomes. The bio-ink formulations can also comprise exosomes without the presence of the stem cells. Also, disclosed herein is a process for obtaining the bio-ink formulation. Further, the present disclosure provides a bio-printed corneal lenticule comprising the bio-ink formulation as described herein. The bio-printed corneal lenticule obtained from the above-mentioned bio-ink formulation provides a controlled swelling, and a high tensile strength. Further, the bio-printed corneal lenticule is also resistant to bio-degradation (2-40% within 30 days under in-vitro conditions) and exhibits transmittance of more than 93%. The bio-printed corneal lenticule thus obtained promotes the growth of stem cells (bio-compatible), and also promotes epithelialization and stromal regeneration, thus providing an opportunity to heal the scars in the corneal tissue. Also, the bio-printed corneal lenticule and/or the hydrogels suppressed myofibroblast differentiation and hence have the potential to support scar-less wound healing of the corneal tissue. The presence of exosomes in the hydrogels/corneal lenticule further supports the regenerative treatment. Thus, the present disclosure provides the bio-ink formulations and corresponding bio-printed corneal lenticule with desired characteristics of tensile strength, compressive modulus, transmittance, controlled swelling and resistant to degradation which can be used to treat corneal defects in a subject to promote scar-less wound healing.

The bio-ink formulation comprising: a modified hyaluronic acid having a molecular weight in the range of 35-70 kDa, and with a degree of substitution in the range of 30-70%; a modified collagen having a molecular weight in the range of 230-270 kDa, and with a degree of substitution in the range of 20-40%; and gelatin having a bloom value in the range of 175-225, and having a concentration range of 40-80 mg/ml with respect to the bio-ink formulation is also disclosed herein. The bio-ink formulation further comprises a photo-initiator (0.5-1×eosin) for initiating cross-linking of the polymers. A white light of certain intensity is illuminated on the bio-ink to further complete the process of cross-linking. The bio-ink formulations further comprise stem cells selected from the group consisting of human bone marrow-mesenchymal stem cell, adipose tissue-mesenchymal stem cell, umbilical cord-mesenchymal stem cell, Wharton jelly-mesenchymal stem cell, dental pulp-derived mesenchymal stem cell, and corneal limbal stem cell-derived conditioned media primed mesenchymal stem cells. Further, the bio-ink formulations also comprise exosomes selected from the group consisting of corneal stromal stem cell derived-exosomes, primed mesenchymal stem cell derived-exosomes, and naive mesenchymal stem cell derived-exosomes. The bio-ink formulations can also comprise exosomes without the presence of the stem cells. Also, disclosed herein is a process for obtaining the bio-ink formulation. Further, the present disclosure provides a bio-printed corneal lenticule comprising the bio-ink formulation as described herein. The bio-printed corneal lenticule obtained from the above-mentioned bio-ink formulation provides a controlled swelling, and a high tensile strength. Further, the bio-printed corneal lenticule is also resistant to bio-degradation (2-40% within 30 days under in-vitro conditions) and exhibits transmittance of more than 93%. The bio-printed corneal lenticule thus obtained promotes the growth of stem cells (bio-compatible), and also promotes epithelialization and stromal regeneration, thus providing an opportunity to heal the scars in the corneal tissue. Also, the bio-printed corneal lenticule and/or the hydrogels suppressed myofibroblast differentiation and hence have the potential to support scar-less wound healing of the corneal tissue. The presence of exosomes in the hydrogels/corneal lenticule further supports the regenerative treatment. Thus, the present disclosure provides the bio-ink formulations and corresponding bio-printed corneal lenticule with desired characteristics of tensile strength, compressive modulus, transmittance, controlled swelling and resistant to degradation which can be used to treat corneal defects in a subject to promote scar-less wound healing. The bio-printed corneal lenticule as described in the present disclosure has the property in which gelatin is leached out in the in-vitro conditions (in the presence of a buffer or a culture medium) amounting to around 60-65% by weight in 20-25 hours.

1-38. (canceled)

39. A formulation for application to the cornea, the formulation comprising:
a hyaluronic acid;
a gelatin; and
exosomes.

40. The formulation as claimed in claim 39, wherein the exosomes are purified from a secretome.

41. The formulation as claimed in claim 39, wherein the exosomes are selected from the group consisting of naive mesenchymal stem cell-derived exosomes, primed mesenchymal stem cell derived-exosomes, and corneal stromal stem cell derived-exosomes.

42. The formulation as claimed in claim 39, wherein the exosomes are primed mesenchymal stem cell-derived exosomes.

43. The formulation as claimed in claim 42, wherein the primed mesenchymal stem cell-derived exosomes are exosomes derived from mesenchymal stem cells primed with a corneal stromal stem cell derived-conditioned medium.

44. The formulation as claimed in claim 39, wherein the exosomes are present a concentration in the range of 0.5-25 billion exosomes per ml of the formulation.

45. The formulation as claimed in claim 39, the formulation comprising a thickener selected from the group consisting of gelatin, gellan gum, xanthum gum, cellulose derivatives, such as methyl cellulose, carboxymethyl cellulose (CMC), hydroxypropyl methyl cellulose (HPMC) and hydroxyethyl methyl cellulose (HEMC), polyethylene glycol, poloxamer, polyvinyl alcohol, and alginate.

46. The formulation as claimed in claim 39, wherein the hyaluronic acid is present in a concentration range of 2-100 mg/ml with respect to the formulation.

47. The formulation as claimed in claim 39, wherein the hyaluronic acid is a methacrylated hyaluronic acid.

48. The formulation as claimed in claim 39, wherein the hyaluronic acid is a thiolated 49 acid.

49. The formulation as claimed in claim 39, wherein the gelatin is present in a concentration range of 0.1-150 mg/ml with respect to the formulation.

50. The formulation as claimed in claim 39, wherein the gelatin is characterized with a bloom value in the range of 50-325.

51. The formulation as claimed in claim 39, wherein the gelatin is a methacrylated gelatin.

52. The formulation as claimed in claim 39, wherein the gelatin is a thiolated gelatin.

53. The formulation as claimed in claim 39, wherein the formulation comprises a photo-activator.

54. The formulation as claimed in claim 39, wherein the photoactivator is an eosin or a riboflavin.

55. The formulation as claimed in claim 39, wherein the formulation comprises stem cells.

56. The formulation as claimed in claim 55, wherein stem cells are selected from the group consisting of human corneal stromal stem cells, human corneal limbal stem cells, human bone marrow-derived mesenchymal stem cells, adipose tissue-derived mesenchymal stem cells, umbilical cord-derived mesenchymal stem cells, Wharton jelly-derived mesenchymal stem cells, dental pulp derived mesenchymal stem cells, placental mesenchymal stem cells, and induced pluripotent stem cells.

57. The formulation as claimed in claim 56, wherein the stem cells are present in the range of 0.1-100 million cells/ml of the formulation.

58. The formulation as claimed in claim 45, wherein the formulation is characterized with a viscosity in the range of 1690-5300 cP.
